(19) United States
(12) Patent Application Publication
(10) Pub. No.: US 2005/0143350 A1
(43) Pub. Date: Jun. 30, 2005

(54) Combination drug therapy to treat obesity

(76) Inventor: John C. Seed, Princeton, NJ (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/993,496

(22) Filed: Nov. 18, 2004

Related U.S. Application Data
(60) Provisional application No. 60/523,610, filed on Nov. 19, 2003.

(51) Int. Cl. A61K 31/66; A61K 31/55; A61K 31/5415; A61K 31/473; A61K 31/4439; A61K 31/426; A61K 31/407

(52) U.S. Cl. 514/114; 514/225.8; 514/340; 514/411; 514/365; 514/297; 514/214.03

(57) ABSTRACT

Provided are methods of achieving desirable weight loss in an overweight or obese individual by administering at least one anticholinesterase agent and at least one antidepressant. The invention also provides for pharmaceutical compositions and kits for simultaneous delivery of at least one anticholinesterase agent and at least one antidepressant.

### CROSS-REFERENCES TO RELATED APPLICATIONS

This application claims the benefit of U.S. Provisional Patent Application No. 60/523,610, filed on Nov. 19, 2003, the disclosure of which is hereby incorporated herein by reference in its entirety.

### STATEMENT AS TO RIGHTS TO INVENTIONS MADE UNDER FEDERALLY SPONSORED RESEARCH AND DEVELOPMENT

NOT APPLICABLE

### BACKGROUND OF THE INVENTION

Obesity is the most common nutritional disorder in the United States, and perhaps in the developed world. Numerous studies indicate that reducing excessive body weight dramatically decreases the risk for chronic diseases, such as diabetes, hypertension, hyperlipidemia, coronary heart disease, and musculoskeletal diseases. Currently available pharmacological treatments for obesity and weight loss have included administering a selective serotonin reuptake inhibitor (SSRI) together with the anorexiant, phentermine (see, U.S. Pat. No. 6,548,551); administering optically pure sibutramine metabolites, (see, U.S. Pat. No. 6,538,034); and administering reserpine with an antidepressant such as trazodone, bupropion or fluoxetine (see, U.S. Pat. No. 4,895,845). Other pharmacological treatments have included administering an acetylcholine esterase reactivator (see, U.S. Pat. No. 5,900,418), an aza-indolyl derivative (see, U.S. Pat. No. 6,583,134) or compounds that increase thermogenesis and increase lipolysis (see, U.S. Pat. No. 6,534,496).

The problems with current pharmacological treatments for weight loss and obesity include that the medications fail to assist many patients achieve weight loss in the first place. Those pharmacological regimens that initially work often fail to assist many patients to continue to achieve weight loss or to maintain a stable weight. Clearly, there is still a need for efficacious pharmacological treatments for achieving desired weight loss and for treating obesity. The present invention fulfills this and other needs.

### BRIEF SUMMARY OF THE INVENTION

The present invention provides methods for treating obesity, achieving desirable weight loss, preventing undesirable weight gain, facilitating weight loss, assisting weight loss, methods of maintaining a stable weight and methods of reducing body weight in an obese or an overweight individual, the methods generally comprising administering to the individual an effective amount of a combination of one or more cholinesterase inhibitors and one or more antidepressants. In a preferred embodiment, the methods comprise administering to an obese or an overweight individual an effective amount of venlafaxine and rivastigmine. Usually, the methods are carried out over an extended period of time. The invention also provides pharmaceutical compositions comprised of a mixture of one or more cholinesterase inhibitors and one or more antidepressants. In a preferred embodiment, the pharmaceutical compositions comprise controlled release formulations.

### BRIEF DESCRIPTION OF THE DRAWINGS

NOT APPLICABLE

### DETAILED DESCRIPTION OF THE INVENTION

Definitions

The term “obese” or “obesity” refers to an individual who has a body mass index (BMI) of 30 kg/m² or more due to excess adipose tissue. Obesity also can be defined on the basis of body fat content: greater than 25% body fat content for a male or more than 30% body fat content for a female. A “morbidly obese” individual has a body mass index greater than 35 kg/m².

The term “overweight” refers to an individual who has a body mass index of 25 kg/m² or more, but less than 30 kg/m².

The term “body mass index” or “BMI” refers to a weight to height ratio measurement that estimates whether an individual's weight is appropriate for their height. As used herein, an individual's body mass index is calculated as follows:
BMI=(pounds×700)/(height in inches)²
or
BMI=(kilograms)/(height in meters)²

The term “baseline body weight” refers to the body weight presented by the individual at the initiation of treatment.

As used herein, “administering” means oral administration, administration as a suppository, topical contact, intravenous, intraperitoneal, intramuscular, intralesional, intranasal or subcutaneous administration, or the implantation of a slow-release device e.g., a mini-osmotic pump, to a subject. Administration is by any route including parenteral, and transmucosal (e.g., oral, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

The terms “cholinesterase inhibitor” and “anticholinesterase” interchangeably refer to a pharmaceutical compound that inhibits the activity of the enzyme acetylcholinesterase (AChE). Cholinesterase inhibitors are generally classified as “reversible,” “pseudo-irreversible” or “slow reversible,” and “irreversible.” “Reversible” cholinesterase inhibitors typically are non-covalent inhibitors. “Pseudo-irreversible,” “pseudo-reversible” or “slow reversible” cholinesterase inhibitors react covalently or noncovalently with AChE with high affinity. Pseudo-irreversible cholinesterase inhibitors typically, but nonexclusively, have a carbamoyl ester linkage and are hydrolyzed by AChE, but much more slowly than acetylcholine. Attack by the active center serine of AChE gives rise to a carbamoylated AChE. The duration of inhibition by the carbamoylating anticholinesterase agents can be about 3 to 4 hours. The half-life of such carbamoylating agents, for example, physostigmine, neostigmine, and pyridostigmine, can be about 1 to 2 hours. The distinction between “pseudo-irreversible” and “reversible” cholinesterase inhibitors generally reflects quantitative differences in rates of deacylation of the acyl enzyme. With “pseudo-irreversible” cholinesterase inhibitors, the half-life (t_{1/2}) for hydrolysis of the dimethylcarbamoyl enzyme is about 15 to 30 minutes. “Irreversible” cholinesterase inhibitors are usually organophophorus compounds. With “irreversible” cholinesterase inhibitors, the active enzyme can spontaneously regenerate after several hours or so slowly that the return of AChE activity depends on the synthesis of new enzyme. Anticholinesterase agents are well known and discussed in detail in, for example, Goodman and Gilman's The Pharmacological Basis of Therapeutics, Chapter 8, 10^{th} Ed., Hardman, Limbird and Goodman-Gilman, Eds., McGraw-Hill (2001), hereby incorporated herein by reference.

The terms “controlled release,” “sustained release,” “extended release,” and “timed release” are intended to refer interchangeably to any drug-containing formulation in which release of the drug is not immediate, i.e., with a “controlled release” formulation, oral administration does not result in immediate release of the drug into an absorption pool. The terms are used interchangeably with “nonimmediate release” as defined in Remington: The Science and Practice of Pharmacy, ₂₁^{st} Ed., Gennaro, Ed., Lippencott Williams & Wilkins (2003). As discussed therein, immediate and nonimmediate release can be defined kinetically by reference to the following equation:

$$\begin{matrix}Dosage \\ Form\end{matrix} {{\to }_{\begin{matrix}drug \\ release\end{matrix}}}^{{k}_{r}} \begin{matrix}Absorption \\ Pool\end{matrix} {{\to }_{absorption}}^{{k}_{a}} \begin{matrix}Target \\ Area\end{matrix} {{\to }_{elimination}}^{{k}_{e}}$$

The “absorption pool” represents a solution of the drug administered at a particular absorption site, and kᵣ, kₐ and kₑ are first-order rate constants for (1) release of the drug from the formulation, (2) absorption, and (3) elimination, respectively. For immediate release dosage forms, the rate constant for drug release kᵣ is far greater than the absorption rate constant kₐ. For controlled release formulations, the opposite is true, i.e., kᵣ<<kₐ, such that the rate of release of drug from the dosage form is the rate-limiting step in the delivery of the drug to the target area.

The terms “sustained release” and “extended release” are used in their conventional sense to refer to a drug formulation that provides for gradual release of a drug over an extended period of time, for example, 12 hours or more, and that preferably, although not necessarily, results in substantially constant blood levels of a drug over an extended time period.

As used herein, the term “delayed release” refers to a pharmaceutical preparation that passes through the stomach intact and dissolves in the small intestine.

General

The present invention provides an efficacious pharmacological treatment for achieving desired weight loss in an overweight or obese individual, and that effectuates continued weight loss and weight management over an extended period of time. Co-administration of one or more anticholinesterase agents and one or more antidepressant agents unexpectedly provides for maintained weight loss of a greater amount of body weight than is accomplished by administering either category of drug alone, especially in view of the weight gain side-effects commonly associated with the long-term administration of antidepressants (see, for example, Masand and Gupta, Ann. Clin. Psych. 14:175 (2002); and Deshmukh and Franco, Cleve. Clin. J. Med. 70:614 (2003)).

### Detailed Embodiments

Methods of Treating

In one aspect, the present invention provides methods for treating obesity. In another aspect, the invention provides methods of facilitating, assisting and achieving desirable weight loss in an obese or overweight individual. In another aspect the present invention provides methods for reducing body weight in an obese or overweight individual. In another aspect the invention provides for methods for maintaining a stable weight and for preventing undesired weight gain in an obese or overweight individual. Generally, the methods comprise administering to an obese or overweight individual an effective amount of a combination of one or more cholinesterase inhibitors and one or more antidepressants for a period of time effective to produce and/or maintain weight loss.

Usually, the combination of one or more cholinesterase inhibitors and one or more antidepressants are administered to the individual over an extended period of time. Typically, the methods are carried out for at least 20 days, more typically for at least 40, 60, 80 or 100 days, and usually for at least 150, 200, 250, 300, 350 days, 1 year or longer. Certain individuals receive the present treatment methods for longer than a year, typically at least 400, 450, 500, 550, 600, 650, 700, 800, 900, 1000 days, and successfully maintain a lower weight. However, individuals can be treated with the present methods and successfully maintain a lower weight for 2 years, 3 years, 4 years or longer. Importantly, the present methods maintain the desired weight loss and weight stabilization over the extended time period of treatment.

The methods are of use in treating individuals that have not been diagnosed with or are not suffering from depression, but also find use in treating individuals diagnosed with and suffering from depression.

Typically, the anticholinesterase includes one or more of a reversible, or a pseudo-irreversible anticholinesterase. Exemplary reversible inhibitors include tacrine, donepezil and galantamine. Exemplary pseudo-irreversible inhibitors include physostigmine, eptastigmine, pyridostigmine, neostigmine, ganstigmine and rivastigmine. Pseudo-irreversible cholinesterase inhibitors also include carbamate insecticides, including carbaryl (Sevin), propoxur (Baygon), and aldicarb (Temik). Typically, pseudo-irreversible anticholinesterases comprise a carbamate moiety, for example, rivastigmine, eptastigmine, physostigmine, neostigmine, pyridostigmine, and ganstigmine. Other clinically employed reversible anticholinesterase agents suitable for use in the present invention include demecarium, ambenonium, and edrophonium. Additional cholinesterase inhibitors that can find use in the present invention include huperzine A, T-82, phenserine, quilostigmine, and TAK-147. In one preferred embodiment, rivastigmine is administered. In one preferred embodiment, galantamine is administered. In one preferred embodiment, donepezil is administered. Those skilled in the art will readily recognize that other, unlisted anticholinesterase agents are applicable.

In certain embodiments, the anticholinesterase includes one or more of an irreversible anticholinesterase agent. For example, inhibition of cholinesterase activity can be achieved by the use of an organophosphate, including an organofluorophosphate, an organocyanophosphate, an organothiophosphate or an organothiocyanophosphate. Exemplary irreversible inhibitors include sarin, metrifonate, soman, tabun, diisopropyl fluorophosphate (DFP), and the insecticides parathion, paraoxon and malathion. These therapeutic agents covalently modify the cholinesterase by acylation of the active site serine. The half life for action of metrifonate has been reported to be approximately 15 days in humans. Irreversible inhibition can be attractive for improving patient compliance. If necessary, the effects of irreversible cholinesterase inhibitors can be counteracted by giving the patient atropine and or pralidoxime. The former is a nonspecific muscarinic acetylcholine receptor antagonist, and the latter reactivates the cholinesterase by reversing the acylation of the active site serine.

In certain embodiments, the anticholinesterase includes one or more of a cholinesterase inhibitory agent that binds to the acyl pocket of the active center of AChE, the choline subsite of the active center of AChE, or the peripheral anionic site of AChE. For example, edrophonium and tacrine bind to the choline subsite in the vicinity of tryptophan 86 and glutamate 202 of AChE. Donepezil binds with higher affinity to the active center of AChE. Propidium and the peptide toxin fasciculin bind to the peripheral anionic site on AChE. This is reviewed in Goodman and Gilman's The Pharmacological Basis of Therapeutics, supra, at pages 175-89, hereby incorporated herein by reference.

In certain embodiments the anticholinesterase also acts at nicotinic acetylcholine receptors as an allosteric potentiator of their action. An exemplary anticholinesterase that also is a nicotinic receptor potentiator is galantamine.

Administered dosages for anticholinesterase agents and antidepressants are in accordance with dosages and scheduling regimens practiced by those of skill in the art. General guidance for appropriate dosages of all pharmacological agents used in the present methods is provided in Goodman and Gilman's The Pharmacological Basis of Therapeutics, 10^{th} Ed., Hardman, Limbird and Goodman-Gilman, Eds., McGraw-Hill (2001) and in a Physicians' Desk Reference (PDR), for instance, in the 57^{th} or 58^{th} Eds., Thomson PDR (2003 or 2004), each of which is hereby incorporated herein by reference. Published dosages of anticholinesterase agents and antidepressants are for indications distinct from treatments to promote weight loss or inhibit weight gain. Typically, efficacious dosages of anticholinesterase agents and antidepressants for practicing the present invention can be equal to or less than (e.g., about 25, 50, 75 or 100%) the dosages published for other indications, such as for Alzheimer's disease and depression, respectively.

The appropriate dosage of one or more cholinesterase inhibitors will vary according to the chosen route of administration and formulation of the composition, among other factors, such as patient response. The dosage can be increased or decreased over time, as required by an individual patient. Usually, a patient initially is given a low dose, which is then increased to an efficacious dosage tolerable to the patient. For example, effective parenteral doses of neostigmine are from about 0.5 mg to about 2.0 mg per dose and equivalent oral doses are from about 15 to 30 mg per dose or more. Appropriate oral doses of edrophonium chloride are from about 2 mg to about 10 mg per day and oral doses of ambenonium are from about 2.5 mg to about 5 mg per day. Pyridostigmine can be administered in “immediate release” preparations in 30 mg to 60 mg doses and in sustained-release formulations of about 180 mg. For use in carrying out the present methods, rivastigmine can be administered at amount of about 0.4 mg to about 6.0 mg per dose, and usually at about 1.0, 1.5, 2.0, 2.5, 3.0, 4.5 mg per dose, and up to 12.0 mg/day. In the present methods, galantamine can be administered in dosages of about 2-12 mg per day, and usually at about 4, 6, 8 or 10 mg per day. Donepezil can be administered in dosages between about 1 and 10 mg per day, preferably about 5 mg or 10 mg per day.

To provide non-limiting exemplifications, an initial dose of rivastigmine can be 1.25 mg twice a day, for instance, one before breakfast and one before supper (see, PDR, 57th Ed., 2003 (supra)). If the patient loses weight at this dose, the dose is not increased. If weight is not lost, the dose taken before supper can be increased to 2.5 mg. With some patients, a major problem is eating during the evening, i.e., after supper. In this case, the next step would be 1.25 mg administered two to three hours after the before supper dose, instead of increasing the before supper dose to 2.5 mg, for a total daily dose of 4.5 mg. The maximum daily dose is usually 12 mg per day. The total daily dose can be distributed to the patient among the three intervals (morning, supper and evening) according to the patient's needs. If the patient stops the medication for a week or more, treatment can be reinitiated by starting over with a small dose, and the dose can be increased relatively rapidly. Rivastigmine has very few interactions with other drugs since it is not metabolized by the P450 cytochrome. Side effects are usually gastrointestinal and can be handled by adjusting the dose. If needed, a proton pump inhibitor (e.g., lansoprazole, omeprazole) can be used. As another example, an initial dose of galantamine can be 4 mg twice a day taken with breakfast and supper. If this is not effective, the dose can be increased to 8 mg twice a day. The maximum dose is usually 12 mg twice a day. To provide an additional example, donepezil is typically given only once a day, because of its long duration. A starting dose can be 5 mg and the highest dose usually is 10 mg. If a patient cannot tolerate a full dose of a particular cholinesterase inhibitor, a second cholinesterase inhibitor can be given along with the one that is not well tolerated, for instance, a small dose of donepezil plus rivastigmine.

For certain patients, the methods are carried out by first administering an anticholinesterase agent alone and then subsequently co-administering an anticholinesterase and an antidepressant. For certain patients, the methods are carried out by first administering an antidepressant alone and then subsequently co-administering an anticholinesterase and an antidepressant. The patient initially can be given either an antidepressant or an anticholinesterase alone for as long as 3 days, 5 days, 7 days, 10 days, 14 days, 20 days, or 30 days before commencing administration of both an anticholinesterase and an antidepressant. To provide a nonlimiting example, a patient is given venlafaxine alone for a week (7 days) or 10 days and then given both venlafaxine and rivastigmine.

Antidepressant agents for use in the present invention are not limited by their mechanism of action and any class of antidepressant is applicable. For instance, tricyclic antidepressants (TCAs) and analogs thereof, serotonin reuptake inhibitors, monoamine oxidase inhibitors (MAOIs), serotonin agonists and prodrugs thereof, norepinephrine reuptake inhibitors, dopamine reuptake inhibitors, and serotonin reuptake accelerators can all be administered in combination with one or more anticholinesterase agents to effect weight loss or weight stabilization or prevent weight gain. Serotonin reuptake inhibitors include both selective serotonin reuptake inhibitors (SSRIs) and serotonin-norepinephrine reuptake inhibitors (SNRIs). Norepinephrine reuptake inhibitors include both the specific norepinephrine reuptake inhibitors as well as the mixed norepinephrine-dopamine reuptake inhibitors (NDRIs). Serotonin-norepinephrine-dopamine, or “triple reuptake inhibitors” also find use in the present invention.

Tricyclic antidepressants for use in the present invention include amineptine, amitriptyline, clomipramine, desipramine, doxepin, dothiepin, imipramine, nortriptyline, protriptyline, trimipramine, amoxapine, the tetracycle maprotiline and the muscle relaxant cyclobenzaprine. Other unlisted tricyclic antidepressants and analogs thereof can also be used.

In one preferred embodiment, an effective amount of one or more anticholinesterase agents is co-administered with an effective amount of a selective serotonin reuptake inhibitor. Exemplary selective serotonin reuptake inhibitors include citalopram, escitalopram, fluoxetine, fluvoxamine, paroxetine and sertraline, although SSRIs not listed are applicable. In one preferred embodiment, citalopram is co-administered with one or more anticholinesterase agents. In a further preferred embodiment, an effective amount of galantamine is co-administered with an effective amount of citalopram. In a further preferred embodiment, an effective amount of donepezil is co-administered with an effective amount of sertraline.

In one preferred embodiment, an effective amount one or more serotonin-norepinephrine reuptake inhibitors are co-administered with one or more cholinesterase inhibitors. Exemplary serotonin-norepinephrine reuptake inhibitors include milnacipran, mirtazapine, venlafaxine, duloxetine, (−)1-(1-dimethylaminomethyl-5-methoxybenzo-cyclobutan-1-yl) cyclohexanol (S33005), DVS-233 (desvenlafaxine), DVS-233 SR and sibutramine, although SNRIs not listed are also of use. In one preferred embodiment, venlafaxine is co-administered with one or more anticholinesterase agents. In a further preferred embodiment, an effective amount of venlafaxine is co-administered with an effective amount of rivastigmine. In one preferred embodiment, an effective amount of duloxetine is co-administered with an effective amount of one or more anticholinesterase agents.

In other embodiments, an effective amount of one or more selective norepinephrine reuptake inhibitors is co-administered with one or more cholinesterase inhibitors. Exemplary selective norepinephrine reuptake inhibitors include reboxetine and atomoxetine.

In one preferred embodiment, an effective amount of one or more norepinephrine-dopamine reuptake inhibitors are co-administered with one or more cholinesterase inhibitors. Exemplary norepinephrine-dopamine reuptake inhibitors include amineptine, GW353162 and bupropion. In the case of bupropion, metabolites are thought to be responsible for the noradrenergic reuptake blockade.

In one preferred embodiment, an effective amount of one or more triple (serotonin-norepinephrine-dopamine) reuptake inhibitors are co-administered with one or more cholinesterase inhibitors. Exemplary triple reuptake inhibitors include SEP-225289, DOV 216,303 and (+)-1-(3,4-dichlorophenyl)-3-azabicyclo-[3.1.0]hexane hydrochloride (DOV 21,947).

Monoamine oxidase inhibitors for use in the present invention include befloxatone, brofaromine, deprenyl, isocarboxazid, moclobemide, pargyline, phenelzine, selegiline and tranylcypromine, together with their sustained delivery and transdermal delivery forms.

Other antidepressants that can be co-administered with an anticholinesterase agent to effect weight loss or stabilization or prevent weight gain include maprotiline, tianeptine, nefazodone and trazodone.

Appropriate dosages for antidepressants will depend on the chosen route of administration and formulation of the composition, among other factors. For instance, tricyclic antidepressants are administered at a dose of about 25 to about 600 mg/day, and usually at a dose of about 75 to about 300 mg/day. Serotonin-reuptake inhibitors are administered at a dose of about 5 to about 400 mg/day, and usually administered at about 20 to about 250 mg/day. In particular, in practicing the present methods, venlafaxine can be administered at about 9 mg to about 225 mg per dose, and is usually administered at about 37.5 mg, 75 mg, 150 mg or 225 mg per dose. Venlafaxine is typically administered at about 25-550 mg/day and usually at about 37.5-375 mg/day, more typically about 75-225 mg/day, and most typically at about 37.5, 75, 150, 225, or 300 mg/day. As appropriate for an individual patient, daily venlafaxine dosages can be divided and administered one time, two times, three times, four or more times a day. In carrying out the present methods, citalopram is administered at about 5-60 mg/day, and preferably at about 10, 20 or 30 mg/day. Usually, citalopram is administered once a day, for instance in the morning or in the evening. However, some patients are given dosages of citalopram two or more times a day. Atypical antidepressants, including bupropion, nefazodone and trazodone are administered at a dose of about 50-600 mg/day, and usually at about 150-400 mg/day. Monoamine oxidase inhibitors are typically administered at a dose of about 5-90 mg/day, and usually at about 10-60 mg/day.

To provide non-limiting specific examples, a usual dose for the SSRI citalopram is 20 mg per day. It can be given once a day, usually in the morning. It relaxes a patient and makes the decrease in food intake more tolerable. There are no known harmful interactions between citalopram and cholinesterase inhibitors. Citalopram can be used along with venlafaxine. The dose can range from 5 to 60 mg per day. As another example, venlafaxine potentiates the effects of cholinesterase inhibitors. Venlafaxine can initially be administered at 30 to 40 mg per day, with gradual dose increases to 100 to 150 mg per day in a week or two before co-administering one or more cholinesterase inhibitors. If side effects inhibit the use of a larger dose of one or more cholinesterase inhibitors, an increase in the dose of venlafaxine can increase the loss of weight. For the morbidly obese, a dose of 375 mg per day can be used. Venlafaxine is preferably given at the same time as the cholinesterase inhibitors. The absorption of venlafaxine is not influenced by the presence of food. In doses over 300 mg a day, a mild increase in blood pressure may occur in 15% of the patients. This is easily compensated for by the administration of a diuretic, loop diuretic, ACE inhibitor or angiotensin-II receptor type 1 inhibitor or other blood pressure lowering agent. The larger doses of venlafaxine may also cause an increase in heart rate. If this is a problem, the dose of venlafaxine should be lowered. Use of a beta blocking agent is apt to interfere with the therapeutic effects of venlafaxine. Venlafaxine has very little effect on the metabolism of other drugs and other drugs have only a minor effect on the metabolism of venlafaxine.

The combination treatment of the present invention can be administered prophylactically to prevent undesirable weight gain or maintain a stable weight, or therapeutically to achieve a desired weight loss and maintain such weight loss for a sustained period of time. Generally, in practicing the present methods, effective amounts of one or more anticholinesterase agents co-administered with one or more antidepressants can be administered together or separately, simultaneously or at different times. The anticholinesterase agents and the antidepressants independently can be administered once, twice, three, four times daily or more or less often, as needed. Preferably, the one or more anticholinesterase agents and the one or more antidepressants are both administered once daily and at the same time, for instance as an admixture. Preferably, a combination of one or more anticholinesterase agents and one or more antidepressants is administered in a sustained-release formulation.

Usually, subjects treated according to the present invention can lose at least about 10, 15 to 20 pounds after about 50, 60 to 70 days of treatment, at least about 20, 25, 30 to 35 pounds after about 80, 90, 100 to 110 days of treatment, and at least about 35, 40, 45, 50 to 55 pounds after about 200, 300, 350 to 400 days of treatment. Typically, individuals treated according to the present methods can lose at least about 5%, and more usually at least about 10%, 15% or 20% of their baseline body weight, and stably maintain this desired weight loss by carrying out a treatment regimen for 100, 150, 200, 250, 300, 350, 400, 450, 500, 550, 600, 700, 800, 900, 1000 days or more. Importantly, administering an effective amount of one or more cholinesterase inhibitors and an effective amount of one or more antidepressants over an extended period of time facilitates a stable weight status and the prevention of undesired weight gain throughout the extended time period of treatment. The combination treatment of the present invention is particularly appropriate for obese and overweight individuals, but can also be administered to any individual who desires to lose weight, maintain a stable weight or prevent unwanted weight gain.

In some embodiments, an anorexiant is further administered. Exemplified anorexiants include without limitation, amphetamine, methamphetamine, dextroamphetamine, phentermine, benzphetamine, phendimetrazine, phenmetrazine, diethylpropion, mazindol, fenfluramine, and phenylpropanolamine. Mild stimulants can also be further administered. Exemplified stimulants include pseudoephedrine, methyl phenidate and modafinil.

Pharmaceutical Formulations/Routes of Administration

The present invention further provides a pharmaceutical composition comprising a mixture of an effective amount of one or more cholinesterase inhibitors and one or more antidepressants. Generally, the pharmaceutical compositions comprise an anticholinesterase agent selected from the group consisting of a reversible inhibitor, a pseudo-irreversible inhibitor and an irreversible inhibitor of ACHE. In certain embodiments, the pharmaceutical compositions comprise one or more cholinesterase inhibitors that comprise a carbamate moiety. In one embodiment, the pharmaceutical composition comprises one or more anticholinesterase agents selected from the group consisting of rivastigmine, galantamine and donepezil.

In certain embodiments, the pharmaceutical compositions comprise one or more antidepressants that are a selective serotonin reuptake inhibitor (SSRI), a serotonin-norepinephrine reuptake inhibitor (SNRI), a norepinephrine reuptake inhibitor, a dopamine reuptake inhibitor, a norepinephrine-dopamine reuptake inhibitor (NDRI), a serotonin-epinephrine-dopamine reuptake inhibitor, a serotonin reuptake accelerator, a serotonin agonist and prodrugs thereof. In one embodiment, the pharmaceutical composition comprises one or more antidepressants selected from the group consisting of venlafaxine, duloxetine, fluoxetine, citalopram, escitalopram, fluvoxamine, paroxetine, S33005, DVS-233 (desvenlafaxine), DVS-233 SR, bupropion, GW353162 and sertraline.

In one preferred embodiment, the pharmaceutical composition comprises effective amounts of rivastigmine and venlafaxine. In one preferred embodiment, the pharmaceutical composition comprises effective amounts of galantamine and citalopram. In one preferred embodiment, the pharmaceutical composition comprises effective amounts of donepezil and sertraline. In one preferred embodiment the pharmaceutical composition comprises effective amounts of galantamine and paroxetine. In one preferred embodiment the pharmaceutical composition comprises effective amounts of galantamine and duloxetine.

A combination of one or more anticholinesterase agents and one or more antidepressants can be administered to a subject, e.g., a human patient, a domestic animal such as a cat or a dog, independently or together in the form of their pharmaceutically acceptable salts, or in the form of a pharmaceutical composition where the compounds are mixed with suitable carriers or excipient(s) in a therapeutically effective amount, e.g., at doses effective to effect desired weight loss or maintenance or prevent undesired weight gain.

An anticholinesterase-antidepressant combination of this invention can be incorporated into a variety of formulations for therapeutic administration. More particularly, a combination of the present invention can be formulated into pharmaceutical compositions, together or separately, by formulation with appropriate pharmaceutically acceptable carriers or diluents, and can be formulated into preparations in solid, semi-solid, liquid or gaseous forms, such as tablets, capsules, pills, powders, granules, dragees, gels, slurries, ointments, solutions, suppositories, injections, inhalants and aerosols. As such, administration of an anticholinesterase-antidepressant combination can be achieved in various ways, including oral, buccal, parenteral, intravenous, intradermal (e.g., subcutaneous, intramuscular), transdermal, etc., administration. Moreover, the compound can be administered in a local rather than systemic manner, for example, in a depot or sustained release formulation. In a preferred embodiment, the invention provides for a pharmaceutical composition comprised of at least one anticholinesterase agent and at least one antidepressant.

Suitable formulations for use in the present invention are found in Remington: The Science and Practice of Pharmacy, 21^{st} Ed., Gennaro, Ed., Lippencott Williams & Wilkins (2003), which is hereby incorporated herein by reference. The pharmaceutical compositions described herein can be manufactured in a manner that is known to those of skill in the art, i.e., by means of conventional mixing, dissolving, granulating, dragee-making, levigating, emulsifying, encapsulating, entrapping or lyophilizing processes. The following methods and excipients are merely exemplary and are in no way limiting.

In one preferred embodiment, an anticholinesterase-antidepressant combination is prepared for delivery in a sustained-release, controlled release, extended-release, timed-release or delayed-release formulation, for example, in semipermeable matrices of solid hydrophobic polymers containing the therapeutic agent. Various types of sustained-release materials have been established and are well known by those skilled in the art. Current extended-release formulations include film-coated tablets, multiparticulate or pellet systems, matrix technologies using hydrophilic or lipophilic materials and wax-based tablets with pore-forming excipients (see, for example, Huang, et al. Drug Dev. Ind. Pharm. 29:79 (2003); Pearnchob, et al. Drug Dev. Ind. Pharm. 29:925 (2003); Maggi, et al. Eur. J. Pharm. Biopharm. 55:99 (2003); Khanvilkar, et al., Drug Dev. Ind. Pharm. 228:601 (2002); and Schmidt, et al., Int. J. Pharm. 216:9 (2001)). Sustained-release delivery systems can, depending on their design, release the compounds over the course of hours or days, for instance, over 4, 6, 8, 10, 12, 16, 20, 24 hours or more. Usually, sustained release formulations can be prepared using naturally-occurring or synthetic polymers, for instance, polymeric vinyl pyrrolidones, such as polyvinyl pyrrolidone (PVP); carboxyvinyl hydrophilic polymers; hydrophobic and/or hydrophilic hydrocolloids, such as methylcellulose, ethylcellulose, hydroxypropylcellulose, and hydroxypropylmethylcellulose; and carboxypolymethylene.

The sustained or extended-release formulations can also be prepared using natural ingredients, such as minerals, including titanium dioxide, silicon dioxide, zinc oxide, and clay (see, U.S. Pat. No. 6,638,521, herein incorporated by reference). Exemplified extended release formulations that can be used in delivering an anticholinesterase-antidepressant combination of the present invention include those described in U.S. Pat. Nos. 6,635,680; 6,624,200; 6,613,361; 6,613,358, 6,596,308; 6,589,563; 6,562,375; 6,548,084; 6,541,020; 6,537,579; 6,528,080 and 6,524,621, each of which is hereby incorporated herein by reference. Controlled release formulations of particular interest include those described in U.S. Pat. Nos. 6,607,751; 6,599,529; 6,569,463; 6,565,883; 6,482,440; 6,403,597; 6,319,919; 6,150,354; 6,080,736; 5,672,356; 5,472,704; 5,445,829; 5,312,817 and 5,296,483, each of which is hereby incorporated herein by reference. Those skilled in the art will readily recognize other applicable sustained release formulations.

For oral administration, an anticholinesterase-antidepressant combination can be formulated readily by combining with pharmaceutically acceptable carriers that are well known in the art. Such carriers enable the compounds to be formulated as tablets, pills, dragees, capsules, emulsions, lipophilic and hydrophilic suspensions, liquids, gels, syrups, slurries, suspensions and the like, for oral ingestion by a patient to be treated. Pharmaceutical preparations for oral use can be obtained by mixing the compounds with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents can be added, such as a cross-linked polyvinyl pyrrolidone, agar, or alginic acid or a salt thereof such as sodium alginate.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

Dragee cores are provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

The compounds can be formulated for parenteral administration by injection, e.g., by bolus injection or continuous infusion. For injection, an anticholinesterase-antidepressant can be formulated into preparations by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. Preferably, a combination of the invention can be formulated in aqueous solutions, preferably in physiologically compatible buffers such as Hanks's solution, Ringer's solution, or physiological saline buffer. Formulations for injection can be presented in unit dosage form, e.g., in ampules or in multi-dose containers, with an added preservative. The compositions can take such forms as suspensions, solutions or emulsions in oily or aqueous vehicles, and can contain formulatory agents such as suspending, stabilizing and/or dispersing agents.

Pharmaceutical formulations for parenteral administration include aqueous solutions of the active compounds in water-soluble form. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. Optionally, the suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

Systemic administration can also be by transmucosal or transdermal means. For transmucosal or transdermal administration, penetrants appropriate to the barrier to be permeated are used in the formulation. For topical administration, the agents are formulated into ointments, creams, salves, powders and gels. In one embodiment, the transdermal delivery agent can be DMSO. Transdermal delivery systems can include, e.g., patches. For transmucosal administration, penetrants appropriate to the barrier to be permeated are used in the formulation. Such penetrants are generally known in the art. Exemplified transdermal delivery formulations that can find use in the present invention include those described in U.S. Pat. Nos. 6,589,549; 6,544,548; 6,517,864; 6,512,010; 6,465,006; 6,379,696; 6,312,717 and 6,310,177, each of which are hereby incorporated herein by reference.

For buccal administration, the compositions can take the form of tablets or lozenges formulated in conventional manner.

In addition to the formulations described previously, an anticholinesterase-antidepressant combination of the present invention can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also can comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Pharmaceutical compositions suitable for use in the present invention include compositions wherein the active ingredients are contained in a therapeutically effective amount. The amount of composition administered will, of course, be dependent on the subject being treated, on the subject's weight, the severity of the affliction, the manner of administration and the judgment of the prescribing physician. Determination of an effective amount is well within the capability of those skilled in the art, especially in light of the detailed disclosure provided herein. Generally, an efficacious or effective amount of a combination of one or more anticholinesterase agents and one or more antidepressants is determined by first administering a low dose or small amount of an anticholinesterase agent alone, an antidepressant alone or a combination of an anticholinesterase agent and an antidepressant, and then incrementally increasing the administered dose or dosages, adding the second medication as needed, until a desired effect of weight loss or stability or prevention of weight gain is observed in the treated subject, with minimal or no toxic side effects. Applicable methods for determining an appropriate dose and dosing schedule for administration of a combination of the present invention are described, for example, in Goodman and Gilman's The Pharmacological Basis of Therapeutics, 10^{th} Ed., Hardman, Limbird and Goodman-Gilman, Eds., McGraw-Hill (2001), and in Remington: The Science and Practice of Pharmacy, 21^{th} Ed., Gennaro, Ed., Lippencott Williams & Wilkins (2003), both of which are hereby incorporated herein by reference.

Dosage amount and interval can be adjusted individually to provide plasma levels of the active compounds which are sufficient to maintain therapeutic effect. Preferably, therapeutically effective serum levels will be achieved by administering single daily doses, but efficacious multiple daily dose schedules are included in the invention. In cases of local administration or selective uptake, the effective local concentration of the drug may not be related to plasma concentration. One having skill in the art will be able to optimize therapeutically effective local dosages without undue experimentation.

The pharmaceutical compositions of the present invention can be provided in a kit. In certain embodiments, a kit of the present invention comprises one or more anticholinesterase agents and one or more antidepressants in separate formulations. In certain embodiments, the kits comprise one or more anticholinesterase agents and one or more antidepressants within the same formulation. In certain embodiments, the kits provide the one or more anticholinesterase agents and one or more antidepressants in uniform dosage formulations throughout the course of treatment. In certain embodiments, the kits provide the one or more anticholinesterase agents and one or more antidepressants in graduated dosages over the course of treatment, either increasing or decreasing, but usually increasing to an efficacious dosage level, according to the requirements of an individual.

In one embodiment, the kits comprise one or more pharmaceutical compositions comprising one or more anticholinesterase agents selected from the group consisting of a reversible inhibitor, a pseudo-irreversible inhibitor, and an irreversible inhibitor. In one embodiment, the kits comprise one or more pharmaceutical compositions comprising one or more anticholinesterase agents selected from the group consisting of rivastigmine, galantamine and donepezil.

In certain embodiments, the kits comprise one or more antidepressants selected from the group consisting of a selective serotonin reuptake inhibitor (SSRI), a serotonin-norepinephrine reuptake inhibitor (SNRI), an epinephrine reuptake inhibitor, a dopamine reuptake inhibitor, a norepinephrine-dopamine reuptake inhibitor (NDRI), a serotonin-norepinephrine-dopamine reuptake inhibitor, and mixtures thereof. In one embodiment, the kits comprise one or more pharmaceutical compositions comprising one or more antidepressants selected from the group consisting of venlafaxine, duloxetine, paroxetine, citalopram, escitalopram, fluvoxamine, S33005, DVS-233 (desvenlafaxine), DVS-233 SR, bupropion, GW353162 and sertraline.

In one preferred embodiment, the kits comprise one or more pharmaceutical compositions comprising effective amounts of rivastigmine and venlafaxine. In one preferred embodiment, the kits comprise one or more pharmaceutical compositions comprising effective amounts of galantamine and citalopram. In one preferred embodiment, the kits comprise one or more pharmaceutical compositions comprising effective amounts of donepezil and sertraline. In one preferred embodiment, the kits comprise one or more pharmaceutical compositions comprising effective amounts of galantamine and paroxetine. In one preferred embodiment, the kits comprise one or more pharmaceutical compositions comprising effective amounts of galantamine and duloxetine.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims. The invention will be described in greater detail by way of specific examples.

The following examples are offered for illustrative purposes, and are not intended to limit the invention in any manner. Those of skill in the art will readily recognize a variety of noncritical parameters which can be changed or modified to yield essentially the same results.

### EXAMPLES

The following examples illustrate exemplified treatment regimens and resultant synergistic effects in achieving long-term stable weight loss by co-administering to an individual an anticholinesterase agent and an antidepressant. Each patient is given an alphanumeric designator.

| | | | | | | | | | | | | | | Traza- | | | |
|---|---|---|---|---|---|---|---|---|---|---|---|---|---|---|---|---|---|
| Patient | | | Wt. | | a | | a | | | a | | a | a | done | | Diethyl- | |
| A1 | | Weight | Chg. | Days | brkfast | | lunch | bedtime | | brkfast | | lunch | supper | am | pm | propion | Comments |
| | | | | | Citalopram | | | | | Galantamine | | | | | | | |
| Height | 63.00 | 167.0 | 0.0 | 0 | | | | | | | | | | | | | |
| BMI | 29.6 | 165.3 | 1.7 | 18 | | | | 20 | mg | 4 | mg | | | | | | |
| Sex | M | 158.5 | 8.5 | 30 | | | | 20 | | 4.0 | | | | | | | |
| Birth Date | Jul. 24, 1951 | 156.5 | 10.5 | 44 | | | | 20 | | 4.0 | | | | | | | |
| Age at onset | 51 | 152.5 | 14.5 | 65 | | | | 20 | | 4.0 | | | | | | | |
| Initial info. | | 152.0 | 15.0 | 93 | | | | 20 | | 4.0 | | | | | | | |
| B.P. | 120/78 | 152.75 | 14.3 | 109 | | | | 20 | | 4.0 | | | 4.0 | | | | |
| Pulse | 64 | | | | Venlafaxine | | | | | Rivastigmine | | | | | | | |
| HPN Rx | NO | | | 116 | | | | | | 1.5 | | | 1.5 | | | | |
| | | 152.25 | 14.8 | 123 | | | | | | 0 | | | 0 | | | | |
| T. Chol. | 212 | | | 135 | 75 | mg | | 75 | mg | 1.5 | | | 1.5 | | | | |
| HDL | 63 | 153.0 | 14.0 | 137 | 0 | | | 0 | | 1.5 | | | 1.5 | | | | |
| TRG. | 55 | 151.75 | 15.3 | 151 | 75 | | | 75 | | 1.5 | | | 1.5 | | | | |
| Chol Rx | NO | 150.25 | 16.8 | 165 | 75 | | | 75 | | 1.5 | | | 1.5 | | | | |
| | | | | 172 | 75 | | | 75 | | 1.5 | | | 1.5 | | | | |
| Athero . . . | NO | 152.75 | 14.3 | 179 | 0 | | | 0 | | 1.5 | | | 1.5 | | | | |
| | | 150.8 | 16.0 | 186 | 0 | | | 0 | | 1.5 | | | 1.5 | | | | |
| Diabetes 2 | NO | 149 | 18.0 | 221 | 75 | | | 75 | | 0 | | | 0 | | | | |
| Diab. Rx | NO | 150.25 | 16.8 | 242 | 75 | | | 75 | | 1.5 | | | 1.5 | | | | |
| | | 150.5 | 16.5 | 249 | 75 | XR | | 75 | XR | 0.75 | | | 0.75 | | | | Was not |
| | | | | | | | | | | | | | | | | | taking IR |
| Depress | NO | 152.0 | 15.0 | 270 | 75 | | | 75 | | 0.75 | | | 0.75 | | | | |
| Depress Rx | NO | 156.0 | 11.0 | 341 | | | | | | | | | | | | | |
| Medications | | | | | | | | | | | | | | | | | |
| Allergy meds | | | | | | | | | | | | | | | | | |
| psoriasis meds | | | | | | | | | | | | | | | | | |

| | | | | | Venlafaxine | | | | | Rivastigmine | | | | | Traza- | | | |
|---|---|---|---|---|---|---|---|---|---|---|---|---|---|---|---|---|---|---|
| Patient | | | Wt. | | a | | a | a | | a | | a | a | | done | | Diethyl- | |
| B1 | | Weight | Chg. | Days | brkfast | | lunch | supper | | brkfast | | lunch | supper | | am | pm | propion | Comments |
| Height | 65.75 | 251.0 | 0.0 | −125 | | | | | | | | | | | | | | |
| BMI | 42.7 | 264.5 | −13.5 | −36 | | | | | | | | | | | | | | |
| Sex | F | 265.0 | −14.0 | −15 | | | | | | | | | | | | | | |
| Birth Date | Jan. 12, | 270.0 | 0.0 | 0 | 150 | mg | | 150 | mg | 3 | mg | | 3 | mg | | | | |
| | 1940 | | | | | | | | | | | | | | | | | |
| Age at onset | 63 | 263.3 | 6.7 | 23 | 225 | | | 225 | | 4.5 | | | 4.5 | | | | | |
| Initial info. | | 261.8 | 8.2 | 37 | 225 | | | 225 | | 4.5 | | | 4.5 | | | | | |
| B.P. | 124/70 | 255.5 | 14.5 | 59 | 225 | | | 225 | | 4.5 | | | 4.5 | | | | | Sweating and |
| | | | | | | | | | | | | | | | | | | uncomfortable |
| | | | | | | | | | | | | | | | | | | stomach in |
| | | | | | | | | | | | | | | | | | | a.m. |
| Pulse | 60 | 261.0 | 9.0 | 79 | 225 | | | 225 | | 4.5 | | | 4.5 | | | | | |
| HPN Rx | YES | 257.0 | 13.0 | 100 | 150 | | | 150 | | 4.5 | | | 4.5 | | | | | |
| | | 254.5 | 15.5 | 163 | 0 | | | 0 | | 0.0 | | | 0.0 | | | | | |
| T. Chol. | 181 | 267.2 | 2.8 | 351 | | | | 0 | | | | | 0.0 | | | | | |
| HDL | 49 | 263.25 | 6.6 | 372 | | | | 200 | | | | | 3.0 | | | | | |
| TRG. | 123 | 265.25 | 4.8 | 393 | | | | 200 | | | | | 3.0 | | | | | |
| Chol Rx | NO | 263.5 | 6.5 | 420 | | | | 75 | | | | | 1.5 | | | | | |
| | | 260.5 | 9.5 | 436 | | | | 0 | | | | | 0.0 | | | | | |
| Athero . . . | | 262.5 | 7.5 | 463 | | | | 0 | | | | | 0.0 | | | | | |
| Diabetes 2 | YES | | | | | | | | | | | | | | | | | |
| Diab. Rx | | | | | | | | | | | | | | | | | | |
| Depress | NO | | | | | | | | | | | | | | | | | |
| Depress Rx | | | | | | | | | | | | | | | | | | |
| Medications | | | | | | | | | | | | | | | | | | |
| HCTZ | | | | | | | | | | | | | | | | | | |
| Atenolol | | | | | | | | | | | | | | | | | | |
| Hydralazine | | | | | | | | | | | | | | | | | | |
| Lisinopril | | | | | | | | | | | | | | | | | | |
| Rosiglitazone | | | | | | | | | | | | | | | | | | Metformin |
| | | | | | | | | | | | | | | | | | | causes |
| | | | | | | | | | | | | | | | | | | diarrhea |
| Atorvastatin | | | | | | | | | | | | | | | | | | |
| Levothyroxine | | | | | | | | | | | | | | | | | | |
| doxazosin | | | | | | | | | | | | | | | | | | |
| metformin | | | | | | | | | | | | | | | | | | |
| alprazolam | | | | | | | | | | | | | | | | | | |
| candesartan | | | | | | | | | | | | | | | | | | |

| | | | | | Venlafaxine | | | | | Rivastigmine | | | | | Traza- | | | |
|---|---|---|---|---|---|---|---|---|---|---|---|---|---|---|---|---|---|---|
| Patient | | | Wt. | | a | | a | a | | a | | a | a | | done | | Diethyl- | |
| B2 | | Weight | Chg. | Days | brkfast | | lunch | supper | | brkfast | | lunch | supper | | am | pm | propion | Comments |
| Height | 68.5 | 205.0 | 0.0 | 0 | | | | | | | | | | | | | | Treatment |
| BMI | 30.8 | 200.0 | 5.0 | 14 | 75 | mg | | 75 | mg | 1.5 | mg | | 1.5 | mg | | | | by phone |
| Sex | F | 193.0 | 12.0 | 42 | 75 | | | 75 | | 1.5 | | | 1.5 | | | | | |
| Birth Date | Feb. 02, 1956 | 185.0 | 20.0 | 56 | 75 | | | 75 | | 1.5 | | | 1.5 | | | | | |
| Age at onset | 47 | 184.0 | 21.0 | 70 | 75 | | | 75 | | 1.5 | | | 1.5 | | | | | |
| Initial info. | | | | 78 | 75 | | | 75 | | 1.5 | | | 1.5 | | | | | |
| B.P. | | 175.0 | 30.0 | 98 | 75 | | | 75 | | 3.0 | | | 3.0 | | | | | |
| Pulse | | 171.0 | 34.0 | 112 | 75 | | | 75 | | 3.0 | | | 3.0 | | | | | |
| HPN Rx | | 169.0 | 36.0 | 126 | 75 | | | 8/75/10 | | 3.0 | | | 8/3.0/10 | | | | | |
| | | 166.0 | 39.0 | 140 | 75 | | | 75 | | 3.0 | | | 3.0 | | | | | |
| T. Chol. | | 173.0 | 32.0 | 154 | 75 | | | 75 | | 3.0 | | | 3.0 | | | | | Lost Interest |
| HDL | | | | | | | | | | | | | | | | | | |
| TRG. | | | | | | | | | | | | | | | | | | |
| Chol Rx | | | | | | | | | | | | | | | | | | |
| Athero. . | | | | | | | | | | | | | | | | | | |
| Diabetes 2 | | | | | | | | | | | | | | | | | | |
| Diab. Rx | | | | | | | | | | | | | | | | | | |
| Depress | | | | | | | | | | | | | | | | | | |
| Depress Rx | | | | | | | | | | | | | | | | | | |
| Medications | | | | | | | | | | | | | | | | | | |
| Zithromax | | | | | | | | | | | | | | | | | | |

| | | | | | Venlafaxine | | | | | Rivastigmine | | | | | | Traza- | | | |
|---|---|---|---|---|---|---|---|---|---|---|---|---|---|---|---|---|---|---|---|
| Patient | | | | | a | | a | a | | a | | a | | a | | done | | Diethyl- | |
| B3 | | Weight | Δ Wt. | Days | brkfast | | lunch | supper | | brkfast | | lunch | | supper | | am | pm | propion | Comments |
| Height | 71 | 239.75 | 0.0 | 0 | | | | | | | | | | | | | | | |
| BMI | 33.5 | 227.75 | 12.0 | 14 | 75 | mg | | | | 1.5 | mg | | | | | | | ? | |
| Sex | M | 224.25 | 15.5 | 27 | 75 | | | | | 1.5 | | | | 1.5 | mg | | | | |
| Birth | May 06, | 222.75 | 17.0 | 42 | | | | | | 1.5 | | | | 1.5 | | | | | |
| Date | 1924 | | | | | | | | | | | | | | | | | | |
| Age at | 78 | 223.50 | 16.3 | 56 | | | | | | 1.5 | | | | 1.5 | | | | | |
| onset | | | | | | | | | | | | | | | | | | | |
| Initial | | 224.50 | 15.3 | 70 | 75 | | | 75 | mg | 3.0 | | | | 3.0 | | | | | Epigastric |
| info. | | | | | | | | | | | | | | | | | | | discomfort, |
| | | | | | | | | | | | | | | | | | | | no vomiting. |
| B.P. | 116/62 | 218.25 | 21.5 | 84 | | | | 150 | | 1.5 | | | | 3.0 | | | | | |
| Pulse | 70 | 216.25 | 23.5 | 126 | 75 | | | 150 | | 1.5 | | | | 3.0 | | | | | Bowels a |
| | | | | | | | | | | | | | | | | | | | little |
| | | | | | | | | | | | | | | | | | | | loose |
| HPN Rx | YES | 217.25 | 22.5 | 140 | 75 | | | 150 | | 3.0 | | | | 4.5 | | | | | |
| | | 220.00 | 19.8 | 161 | 75 | | | 150 | | 3.0 | | | | 3.0 | | | | 25 mg @ | |
| | | | | | | | | | | | | | | | | | | supper | |
| T. Chol. | 138 | 213.75 | 26.0 | 175 | 75 | | | 225 | | 3.0 | | | | 6.0 | | | | 25 | Vomiting |
| | | | | | | | | | | | | | | | | | | | after |
| | | | | | | | | | | | | | | | | | | | rivastig- |
| | | | | | | | | | | | | | | | | | | | mine at |
| | | | | | | | | | | | | | | | | | | | supper |
| HDL | 44 | 213.75 | 26.0 | 189 | 75 | | | 225 | | 3.0 | | | | 4.5 | | | | | Diethyl- |
| | | | | | | | | | | | | | | | | | | | propion |
| | | | | | | | | | | | | | | | | | | | made him |
| | | | | | | | | | | | | | | | | | | | vomit |
| | | | | | | | | | | | | | | | | | | | twice. |
| TRG. | 76 | 213.25 | 26.5 | 231 | 75 | | | 225 | | 3.0 | | | | 4.5 | | | | | Total |
| | | | | | | | | | | | | | | | | | | | cholesterol |
| | | | | | | | | | | | | | | | | | | | 125 |
| Chol Rx | YES | 215.75 | 24.0 | 245 | 150 | | | 225 | | 6.0 | | | | 6.0 | | | | | |
| | | 223.50 | 16.3 | 259 | 0 | | | 0 | | 0 | | | | 0 | | | | | Stopped |
| | | | | | | | | | | | | | | | | | | | medicine |
| | | | | | | | | | | | | | | | | | | | for 7 day |
| | | | | | | | | | | | | | | | | | | | cruise. |
| Athero . . . | YES | 218.25 | 21.5 | 264 | 75 | | | 150 | | 3.0 | | | | 3.0 | | | | | |
| | | 223.00 | 16.8 | 273 | 75 | | | 150 | | 3.0 | | | | 3.0 | | | | | |
| Diabetes | YES | 220.00 | 19.8 | 277 | | | 75 | 150 | | | | 1.5 | mg | 3.0 | | | | | |
| 2 | | | | | | | | | | | | | | | | | | | |
| Diab. Rx | YES | 217.75 | 22.0 | 301 | | | 75 | 150 | | | | 1.5 | | 4.5 | | | | | |
| | | 222.25 | 17.5 | 315 | | | 0 | 150 | | | | 0 | | | | | | | |
| Depress | | 223.00 | 16.8 | 329 | | | 75 | 150 | | | | 1.5 | | 4.5 | | | | | |
| Depress | YES? | 221.00 | 18.8 | 357 | | | 75 | 150 | | | | 1.5 | | 4.5 | | | | 12.5 | |
| Rx | | | | | | | | | | | | | | | | | | | |
| | | 225.25 | 14.5 | 371 | | | 75 | 150 | | | | 3.0 | | 4.5 | | | | | |
| Medica- | | 225.25 | 14.5 | 385 | 75 | | 75 | 150 | | | | 3.0 | | 4.5 | | | | 25 | |
| tions | | | | | | | | | | | | | | | | | | | |
| Metformin | | 225.50 | 14.3 | 399 | 75 | | 75 | 150 | | | | 3.0 | | 4.5 | | 50 | | 25 | |
| Pioglita- | | 222.25 | 17.5 | 416 | 75 | | 75 | 150 | | | | 3.0 | | 4.5 | | 50 | | 25 | |
| zone | | | | | | | | | | | | | | | | | | | |
| HCTZ | | 225.50 | 14.3 | 441 | 75 | | 75 | 150 | | | | 3.0 | | 3.0 | | | | 25 | broke |
| | | | | | | | | | | | | | | | | | | | wrist |
| Atenolol | | 225.25 | 14.5 | 455 | 75 | | 75 | 150 | | 1.5 | | 3.0 | | 3.0 | | | | 25 | |
| Lansopra- | | 222.75 | 17.0 | 469 | 75 | | 75 | 150 | | 1.5 | | 3.0 | | 4.5/1.5 | | | | cranberry | Donep 5 |
| zole | | | | | | | | | | | | | | | | | | juice | |
| Rosuva- | | 226.20 | 13.55 | 483 | 75 | | 75 | 150 | | 1.5 | | 3.0 | | 4.5 | | | | 25 | Donep 5 |
| statin | | | | | | | | | | | | | | | | | | | |

| Feno- | 225.80 | 13.95 | 485 | 75 | 75 | 150 | 1.5 | 3.0 | 3.0/1.5 | 25 | Low |
|---|---|---|---|---|---|---|---|---|---|---|---|
| fibrate | | | | | | | | | | | glycemic |
| | 213.00 | 26.75 | 511 | 75 | 75 | 150 | 1.5 | 3.0 | 3.0/1.5 | 25 | metform |
| | | | | | | | | | | | 4000 mg |
| | 215.20 | 24.55 | 539 | 75 | 75 | 150 | 1.5 | 3.0 | 3.0/1.5 | 25 | birthday |
| | 213.20 | 26.55 | 553 | 75 | 75 | 150 | 1.5 | 3.0 | 3.0/1.5 | 25 | |
| | 208.80 | 30.95 | 567 | 75 | 75 | 150 | 1.5 | 3.0 | 4.5/1.5 | | |
| | 209.00 | 30.75 | 581 | 75 | 75 | 150 | 1.5 | 3.0 | 4.5/1.5 | | |
| | 209.50 | 30.25 | 595 | 75 | 75 | 150 | 1.5 | 3.0 | 4.5/1.5 | | |
| | 207.50 | 32.25 | 609 | | | | | | | | |
| | 203.50 | 36.25 | 623 | | | | | | | | |

| | | | | | Venlafaxine | | | | | Rivastigmine | | | | | Traza- | | | |
|---|---|---|---|---|---|---|---|---|---|---|---|---|---|---|---|---|---|---|
| Patient | | | Wt. | | a | | a | a | | a | | a | a | | done | | Diethyl- | |
| B4 | | Weight | Chg. | Days | brkfast | | lunch | supper | | brkfast | | lunch | supper | | am | pm | propion | Comments |
| Height | 73 | 264.5 | 0.0 | 0 | | | | | | | | | | | | | | |
| BMI | 35.0 | 254.5 | 10.0 | 30 | 75 | mg | | 75 | mg | 1.5 | mg | | 1.5 | mg | | | | |
| Sex | M | 259.8 | 4.8 | 44 | 75 | | | 75 | | 1.5 | | | 1.5 | | | | | |
| Birth Date | Mar. 11, 1956 | 247.8 | 16.8 | 58 | 75 | | | 150 | | 3.0 | | | 3.0 | | | | | |
| Age at | 47 | 242.5 | 22.0 | 72 | 75 | | | 150 | | 3.0 | | | 3.0 | | | 50 | | |
| onset | | | | | | | | | | | | | | | | | | |
| Initial | | 238.3 | 26.3 | 86 | 75 | | | 150 | | 3.0 | | | 3.0 | | | 50 | | |
| info. | | | | | | | | | | | | | | | | | | |
| B.P. | 118/88 | 243.25 | 21.3 | 98 | 75 | | | 150 | | 3.0 | | | 3.0 | | | 50 | | |
| Pulse | 98 | 245.25 | 19.3 | 114 | 75 | | 75 | 150 | | 4.5 | | | 4.5 | | | 50 | | 0 |
| HPN Rx | YES | 236.75 | 27.8 | 127 | 75 | | 75 | 150 | | 3.0 | | | 4.5 | | | 50 | | 0 |
| T. Chol. | 217 | | | | | | | | | | | | | | | | | |
| HDL | 39 | | | | | | | | | | | | | | | | | |
| TRG. | 217 | | | | | | | | | | | | | | | | | |
| Chol Rx | YES | | | | | | | | | | | | | | | | | |
| Athero . . . | | | | | | | | | | | | | | | | | | |
| Diabetes 2 | | | | | | | | | | | | | | | | | | |
| Diab. Rx | | | | | | | | | | | | | | | | | | |
| Depress | | | | | | | | | | | | | | | | | | |
| Depress Rx | | | | | | | | | | | | | | | | | | |
| Medications | | | | | | | | | | | | | | | | | | |
| Sildenafil | | | | | | | | | | | | | | | | | | |
| Haloperidol | | | | | | | | | | | | | | | | | | |
| Niacin | | | | | | | | | | | | | | | | | | |
| Lisinopril | | | | | | | | | | | | | | | | | | |
| Nasal spray | | | | | | | | | | | | | | | | | | |

| | | | | | Venlafaxine | | | | | Rivastigmine | | | | | Traza- | | | | |
|---|---|---|---|---|---|---|---|---|---|---|---|---|---|---|---|---|---|---|---|
| Patient | | | | | a | | a | a | | a | | a | a | | done | | Diethyl- | | |
| F1 | | Weight | Δ Wt. | Days | brkfast | | lunch | supper | | brkfast | | lunch | supper | | am | pm | propion | | Comments |
| Height | 65.25 | 214.5 | 0.0 | 0 | | | | | | | | | | | | | | | |
| BMI | 35.5 | 208.5 | 6.0 | 7 | 75 | mg | | 75 | mg | | | | | | | | | | |
| Sex | F | 204.3 | 10.3 | 28 | 75 | | | 75 | | 1.25 | mg | | 1.25 | mg | | | | | |
| Birth | Jun. 05, | 204.8 | 9.8 | 43 | 75 | | | 75 | | 1.25 | | | 1.25 | | | | | | |
| Date | 1956 | | | | | | | | | | | | | | | | | | |
| Age at | 46 | 204.8 | 9.8 | 56 | 75 | | | 75 | | 3.0 | | | 3.0 | | | | | | |
| onset | | | | | | | | | | | | | | | | | | | |
| Initial | | | | 72 | 150 | | | 150 | | 3.0 | | | 3.0 | | | | 25 mg | 25 mg | |
| info. | | | | | | | | | | | | | | | | | | | |
| B.P. | 170/100 | 199.8 | 14.8 | 86 | 150 | | | 150 | | 3.0 | | | 3.0 | | | | 25 | 25 | |
| Pulse | 54 | 195.5 | 19.0 | 100 | 150 | | | 150 | | 3.0 | | | 3.0 | | | | 25 | 25 | On Jul. |
| | | | | | | | | | | | | | | | | | | | 3, 2003 |
| | | | | | | | | | | | | | | | | | | | reported |
| | | | | | | | | | | | | | | | | | | | vomitting |
| | | | | | | | | | | | | | | | | | | | everytime |
| | | | | | | | | | | | | | | | | | | | she eats |
| | | | | | | | | | | | | | | | | | | | or drinks |
| | | | | | | | | | | | | | | | | | | | beginning |
| | | | | | | | | | | | | | | | | | | | 6-24. B.P. |
| | | | | | | | | | | | | | | | | | | | 114/78. |
| | | | | | | | | | | | | | | | | | | | HCTZ |
| | | | | | | | | | | | | | | | | | | | stopped |
| HPN Rx | YES | 198.0 | 16.5 | 114 | 150 | | | 150 | | 3.0 | | | 3.0 | | | | 25 | 25 | |
| | | 194.3 | 20.2 | 122 | 150 | | | 150 | | 3.0 | | | 3.0 | | | | 25 | 25 | |
| T. Chol. | 142 | 188.8 | 25.7 | 149 | 150 | | | 150 | | 3.0 | | | 3.0 | | | | 25 | 25 | Feels |
| | | | | | | | | | | | | | | | | | | | good. No |
| | | | | | | | | | | | | | | | | | | | change in |
| | | | | | | | | | | | | | | | | | | | medica- |
| | | | | | | | | | | | | | | | | | | | tions |
| HDL | 45 | 189.0 | 25.5 | 181 | 150 | | | 150 | | 3.0 | | | 3.0 | | | | 25 | 25 | |
| TRG. | 80 | 192.75 | 21.8 | 211 | 0 | | | 0 | | 3.0 | | | 3.0 | | | | | | |
| Chol Rx | YES | 191.0 | 23.5 | 225 | 100 | | | 100 | | 3.0 | | | 3.0 | | | | 0 | 0 | |
| | | 190.0 | 24.5 | 239 | 100 | | | 100 | | 3.0 | | | 3.0 | | | | 25 | 25 | |
| Athero | | 190.50 | 24.0 | 253 | 100 | | | 100 | | 3.0 | | | 3.0 | | | | 25 | 25 | |
| . . . | | 189.75 | 24.8 | 267 | 0 | | | 0 | | 3.0 | | | 3.0 | | | 50 | 0 | 0 | |

| | | | | | | | | | |
|---|---|---|---|---|---|---|---|---|---|
| Diabetes | 191.25 | 23.3 | 281 | 100 | 100 | 3.0 | 1.5 | 3.0/1.5 | 50 |
| 2 | | | | | | | | | |
| Diab. Rx | 183.25 | 31.3 | 302 | 112 | 112 | 3.0 | | 3.0/3.0 | 0 |

| | | | | | | | | | | |
|---|---|---|---|---|---|---|---|---|---|---|
| | 186.75 | 27.75 | 316 | | | | | | | |
| Depress | 185.25 | 29.3 | 330 | | | | | | | |
| Depress | 184.0 | 30.5 | 344 | 75 | 75/75 | 3.0 | | 3.0/3.0 | | |
| Rx | | | | | | | | | | |
| | 177.75 | 36.75 | 358 | 75 | 75/75 | 4.5 | | 4.5/4.5 | | |
| Medica- | 180.0 | 34.5 | 372 | 75 | 75/75 | 4.5 | | 4.5/4.5 | | Desipra- |
| tions | | | | | | | | | | mine, |
| | | | | | | | | | | Funeral |
| HCTZ | 177.0 | 37.5 | 386 | 75 | 75/75 | 4.5 | | 4.5/4.5 | a50 | |
| Meto- | 173.2 | 41.3 | 400 | 75 | 75/75 | 4.5 | | 4.5/4.5 | a50 | |
| prolol | | | | | | | | | | |
| Diltiasem | 176.5 | 38.0 | 414 | 75 | 75/75 | 4.5 | | 4.5/4.5 | a-5, 50 | |
| Spirono- | 175.2 | 39.3 | 428 | 75 | 75/75 | 4.5 | | 4.5/4.5 | a-5, 50 | |
| lactone | | | | | | | | | | |
| Nicorette | 177.5 | 37.0 | 442 | 75 | 150/75 | 4.5 | | 4.5/4.5 | | Ensure, |
| gum | | | | | | | | | | juice |
| Flucona- | 178.0 | 36.5 | 456 | 75 | 150/75 | 4.5 | | 4.5/4.5 | | |
| zole | | | | | | | | | | |
| Cicol- | 179.0 | 35.5 | 470 | 75 | 150/75 | 4.5 | 4.5 | 4.5/4.5 | | |
| spirox | | | | | | | | | | |
| cream | | | | | | | | | | |
| hydrala- | 179.8 | 34.7 | 484 | 75 | 150/75 | 4.5 | 4.5 | 4.5/4.5 | | |
| zine | | | | | | | | | | |
| | 177.0 | 37.5 | 501 | 75 | 150/75 | 4.5 | 4.5 | 4.5/4.5 | | |
| | 174.5 | 40.0 | 512 | 75 | 150/75 | 4.5 | 4.5 | 4.5/4.5 | | |
| | | | | 75 | 150/75 | 4.5 | 4.5 | 4.5/4.5 | | |

| | | | | | Venlafaxine | | | | | Rivastigmine | | | | Traza- | | Diethyl- | | |
|---|---|---|---|---|---|---|---|---|---|---|---|---|---|---|---|---|---|---|
| Patient | | | Wt. | | a | | a | a | | a | | a | a | done | | propion | | |
| H1 | | Weight | Chg. | Days | brkfast | | lunch | supper | | brkfast | | lunch | supper | am | pm | am | pm | Comments |
| Height | 64.75 | 217.8 | 0.0 | 0 | | | | | | | | | | | | | | Last time |
| | | | | | | | | | | | | | | | | | | below 200 Sep. |
| | | | | | | | | | | | | | | | | | | 26, 1995 |
| BMI | 36.9 | 212.3 | 5.5 | 17 | | | | | | 1.5 | mg | | | | | | | Last time |
| | | | | | | | | | | | | | | | | | | below 210 Aug. |
| | | | | | | | | | | | | | | | | | | 08, 2000 |
| Sex | M | 207.3 | 10.5 | 32 | | | | | | 1.5 | | | | | | | | Peak 230.5 Mar. |
| | | | | | | | | | | | | | | | | | | 07, 2000 |
| Birth | May 21, | 209.3 | 8.5 | 37 | | | | | | 1.5 | | | | | | | | |
| Date | 1932 | | | | | | | | | | | | | | | | | |
| Age at | 68 | 211.8 | 6.0 | 52 | | | | | | 1.5 | | | | | | | | |
| onset | | | | | | | | | | | | | | | | | | |
| Initial | | 211.0 | 6.8 | 66 | | | | | | 1.5 | | | 1.5 | | | | | |
| info. | | | | | | | | | | | | | | | | | | |
| B.P. | 134/78 | 207.0 | 10.8 | 84 | | | | | | 1.5 | | | 1.5 | | | | | |
| Pulse | 59 | 204.5 | 13.3 | 101 | | | | | | 1.5 | | | | | | | | |
| HPN Rx | YES | 205.8 | 12.0 | 127 | | | | | | 1.5 | | | 1.5 | | | | | |
| | | 205.8 | 12.0 | 141 | | | | | | 1.5 | | | 1.5 | | | | | |
| T. Chol. | 151 | 205.5 | 12.3 | 157 | | | | | | 1.5 | | | 1.5 | | | | | |
| HDL | 60 | 205.5 | 12.3 | 176 | | | | | | 1.5 | | | 1.5 | | | | | |
| TRG. | 48 | 212.5 | 5.3 | 225 | | | | | | 1.5 | | | 3.0 | | | | | |
| Chol Rx | YES | 212.75 | 5.0 | 232 | | | | | | 1.5 | | | 3.0 | | | | | |
| | | 211.25 | 6.5 | 238 | | | | | | 1.5 | | | 3.0 | | | | | |
| Athero . . . | YES | 208 | 9.8 | 254 | | | | | | 1.5 | | | 3.0 | | | | | |
| | | 219.25 | −1.5 | 597 | | | | | | 1.5 | | | 1.5 | | | | | |
| Diabetes 2 | YES | 215.0 | 2.8 | 611 | 75 | mg | | 75 | mg | 1.5 | | | 1.5 | | | | | |
| Diab. Rx | YES | 215.3 | 2.4 | 626 | 75 | | | 75 | | 3.0 | | | 3.0 | | | | | |
| | | 216.5 | 1.3 | 644 | 75 | | | 75 | | 3.0 | | | 3.0 | | | | | |
| Depress | NO | 218.0 | −0.3 | 661 | 75 | | | 75 | | 3.0 | | | 4.5 | | | | | |
| Depress Rx | | 217.8 | −0.1 | 675 | 150 | | | 75 | | 4.5 | | | 4.5 | | | | | |
| | | 216.5 | 1.3 | 703 | 75 | | | 75 | | 4.5 | | | 4.5 | | | | | Thailand |
| Medications | | 219.5 | −1.8 | 714 | 75 | | | 150 | | 4.5 | | | 4.5 | | | | | |
| Lisinopril | | 215.5 | 2.3 | 826 | 75 | | | 150 | | 6.0 | | | 6.0 | | | | | |
| HCTZ | | 215.5 | 2.3 | 840 | 75 | | | 150 | | 4.5 | | | 4.5 | | | 25 | 25 | |
| Doxazosin | | 220.8 | −3.1 | 843 | 75 | | | 150 | | | | | | | | 25 | 25 | Alaska cruise |
| Atenolol | | 209.0 | 8.8 | 906 | 75 | | | 150 | | 1.5 | | | 3.0 | | | 25 | 25 | |
| Metformin | | 209.0 | 8.8 | 927 | 75 | | 75 | 150 | | 3.0 | | 1.5 | 4.5 | | | 25 | 25 | |
| Alprazolam | | | | | | | | | | | | | | | | | | |
| Paxil | | | | | | | | | | | | | | | | | | |
| glucophage | | | | | | | | | | | | | | | | | | |

| | | | | | Venlafaxine | | | | | Rivastigmine | | | | | Traza- | | | |
|---|---|---|---|---|---|---|---|---|---|---|---|---|---|---|---|---|---|---|
| Patient | | | Wt. | | a | | a | a | | a | | a | a | | done | | Diethyl- | |
| K1 | | Weight | Chg. | Days | brkfast | | lunch | supper | | brkfast | | lunch | supper | | am | pm | propion | Comments |
| Height | 72.0 | | | 0 | | | | | | | | | | | | | | Rosaglitazone, |
| | | | | | | | | | | | | | | | | | | Apr. 11, 1999 |
| BMI | 33.6 | 247.5 | 0.0 | 22 | 150 | mg | | 150 | mg | 3.0 | mg | | 3.0 | mg | | | | |
| Sex | M | 245.0 | 2.5 | 27 | 150 | | | 150 | | 3.0 | | | 3.0 | | | | | |
| Birth | Jun. 16, | 236.3 | 11.3 | 56 | 150 | | | 150 | | 3.0 | | | 3.0 | | | | | |
| Date | 1946 | | | | | | | | | | | | | | | | | |
| Age at | 56 | 232.0 | 15.5 | 71 | 150 | | | 150 | | 3.0 | | | 3.0 | | | | | |
| onset | | | | | | | | | | | | | | | | | | |
| Initial | | 227.8 | 19.8 | 93 | 150 | | | 150 | | 3.0 | | | 3.0 | | | | | |
| info. | | | | | | | | | | | | | | | | | | |
| B.P. | 134/80 | 223.8 | 23.8 | 240 | 150 | | | 150 | | 3.0 | | | 3.0 | | | | | |
| Pulse | 84 | | | 247 | 100 | | | 100 | | 4.5 | | | 4.5 | | | | | |
| HPN Rx | YES | 221.8 | 25.8 | 269 | 100 | | | 100 | | 4.5 | | | 4.5 | | | | | A1C below 6.0 |
| T. Chol. | 144 | | | | | | | | | | | | | | | | | |
| HDL | 39 | | | | | | | | | | | | | | | | | |
| TRG. | 264 | | | | | | | | | | | | | | | | | |
| Chol Rx | YES | | | | | | | | | | | | | | | | | |
| Athero . . . | | | | | | | | | | | | | | | | | | |
| Diabetes 2 | YES | | | | | | | | | | | | | | | | | |
| Diab. Rx | | | | | | | | | | | | | | | | | | |
| Depress | ? | | | | | | | | | | | | | | | | | |
| Depress Rx | | | | | | | | | | | | | | | | | | |
| Medications | | | | | | | | | | | | | | | | | | |
| Metformin | | | | | | | | | | | | | | | | | | |
| Rosiglitazone | | | | | | | | | | | | | | | | | | |
| Glyburide | | | | | | | | | | | | | | | | | | |
| Sildeinafil | | | | | | | | | | | | | | | | | | |
| Niaspan | | | | | | | | | | | | | | | | | | |
| Atorvastatin | | | | | | | | | | | | | | | | | | |
| Lisinopril | | | | | | | | | | | | | | | | | | |
| Hydralazine | | | | | | | | | | | | | | | | | | |
| Psyllium | | | | | | | | | | | | | | | | | | |
| Niacin | | | | | | | | | | | | | | | | | | |
| Viagra | | | | | | | | | | | | | | | | | | |
| Bacitracin | | | | | | | | | | | | | | | | | | |

| | | | | | Venlafaxine | | | | | | Rivastigmine | | | | | | Traza- | | | |
|---|---|---|---|---|---|---|---|---|---|---|---|---|---|---|---|---|---|---|---|---|
| Patient | | | Wt. | | a | | a | | a | | a | | a | | a | | done | | Diethyl- | |
| K2 | | Weight | Chg. | Days | brkfast | | lunch | | supper | | brkfast | | lunch | | supper | | am | pm | propion | Comments |
| Height | 61.50 | 145.5 | 0.0 | 0 | | | | | | | | | | | | | | | | |
| BMI | 27.1 | | | 14 | | | | | | | | | | | | | | | | |
| Sex | F | 132.0 | 13.5 | 19 | 37.5 | mg | 37.5 | mg | 37.5 | mg | 1.5 | mg | 1.5 | mg | 1.5 | mg | | | | |
| | | | | | 37.5 | | 37.5 | | 37.5 | | 1.5 | | 1.5 | | 1.5 | | | | | |
| Birth | Oct. 29, | 137.0 | 8.5 | 78 | 37.5 | | 37.5 | | 37.5 | | 1.5 | | 1.5 | | 1.5 | | | | | |
| Date | 1963 | | | | | | | | | | | | | | | | | | | |
| Age at | 39 | | | | 75 | | 75 | | 75 | | 3.0 | | 1.5 | | 3.0 | | | | | |
| onset | | | | | | | | | | | | | | | | | | | | |
| Initial | | | | | | | | | | | | | | | | | | | | |
| info. | | | | | | | | | | | | | | | | | | | | |
| B.P. | 108/78 | | | | | | | | | | | | | | | | | | | |
| Pulse | 78 | | | | | | | | | | | | | | | | | | | |
| HPN Rx | NO | | | | | | | | | | | | | | | | | | | |
| T. Chol. | 210 | | | | | | | | | | | | | | | | | | | |
| HDL | 68 | | | | | | | | | | | | | | | | | | | |
| TRG. | 83 | | | | | | | | | | | | | | | | | | | |
| Chol Rx | NO | | | | | | | | | | | | | | | | | | | |
| Athero . . . | | | | | | | | | | | | | | | | | | | | |
| Diabetes 2 | NO | | | | | | | | | | | | | | | | | | | |
| Diab. Rx | | | | | | | | | | | | | | | | | | | | |
| Depress | NO | | | | | | | | | | | | | | | | | | | |
| Depress Rx | | | | | | | | | | | | | | | | | | | | |
| Medications | | | | | | | | | | | | | | | | | | | | |
| levothy- | | | | | | | | | | | | | | | | | | | | |
| roxine | | | | | | | | | | | | | | | | | | | | |
| rivastig- | | | | | | | | | | | | | | | | | | | | |
| mine | | | | | | | | | | | | | | | | | | | | |
| ven- | | | | | | | | | | | | | | | | | | | | |
| lafaxine | | | | | | | | | | | | | | | | | | | | |
| fish oil | | | | | | | | | | | | | | | | | | | | |
| chromium | | | | | | | | | | | | | | | | | | | | |
| picolinate | | | | | | | | | | | | | | | | | | | | |
| flax oil | | | | | | | | | | | | | | | | | | | | |
| metroni- | | | | | | | | | | | | | | | | | | | | |
| dazole | | | | | | | | | | | | | | | | | | | | |
| gel | | | | | | | | | | | | | | | | | | | | |

| | | | | | Venlafaxine | | | | | Rivastigmine | | | | | | Traza- | | | |
|---|---|---|---|---|---|---|---|---|---|---|---|---|---|---|---|---|---|---|---|
| Patient | | | | | a | | a | a | | a | | a | | a | | done | | Diethyl- | |
| M1 | | Weight | ΔWt. | Days | brkfast | | lunch | supper | | brkfast | | lunch | | supper | | am | pm | propion | Comments |
| Height | 69.25 | 326.0 | 0.0 | 0 | | | | | | | | | | | | | | | Mar '93 - 248, |
| | | | | | | | | | | | | | | | | | | | No data until |
| | | | | | | | | | | | | | | | | | | | Nov '00 - 312, |
| BMI | 48.0 | | | 7 | 75 | mg | | | | 1.5 | mg | | | 1.5 | mg | | | | Never below |
| | | | | | | | | | | | | | | | | | | | 300 until |
| | | | | | | | | | | | | | | | | | | | June '03 |
| Sex | M | 318.5 | 7.5 | 13 | 75 | | | 75 | mg | 1.5 | | | | 1.5 | | | | | |
| Birth | Nov. 03, | 314.5 | 11.5 | 28 | 75 | | | 75 | | 1.5 | | | | 1.5 | | | | | |
| Date | 1957 | | | | | | | | | | | | | | | | | | |
| Age at | 45 | 312.5 | 13.5 | 42 | 75 | | | 75 | | 1.5 | | | | 1.5 | | | | | Skips the |
| onset | | | | | | | | | | | | | | | | | | | evening dose |
| | | | | | | | | | | | | | | | | | | | if his appe- |
| | | | | | | | | | | | | | | | | | | | tite is poor |
| Initial | | 311.8 | 14.3 | 56 | 75 | | | 75 | | 1.5 | | | | 1.5 | | | | | |
| info. | | | | | | | | | | | | | | | | | | | |
| B.P. | 140/100 | 312.3 | 13.8 | 70 | 75 | | | 150 | | 1.5 | | | | 3.0 | | | | | |
| Pulse | 76 | 310.5 | 15.5 | 85 | 150 | | | 150 | | 4.5 | | | | 4.5 | | | | | |
| HPN Rx | YES | 305.0 | 21.0 | 104 | 150 | | | 150 | | 6.0 | | | | 6.0 | | | | | |
| | | 305.0 | 21.0 | 125 | 150 | | | 150 | | 6.0 | | | | 6.0 | | | | | |
| T. Chol. | 183 | 297.8 | 28.2 | 153 | 150 | | | 150 | | 6.0 | | | | 6.0 | | | | | Tried 12 mg |
| | | | | | | | | | | | | | | | | | | | of Riva. |
| | | | | | | | | | | | | | | | | | | | Caused vomit- |
| | | | | | | | | | | | | | | | | | | | ing, diarrhea, |
| | | | | | | | | | | | | | | | | | | | dizzy. B.P. |
| | | | | | | | | | | | | | | | | | | | 88/64 |
| HDL | 61 | 298.0 | 28.0 | 167 | 150 | | | 150 | | 6.0 | | | | 6.0 | | | | | |
| TRG. | 149 | 293.5 | 32.5 | 181 | 150 | | | 150 | | 6.0 | | 6.0 | mg | 6.0 | | | | | Creatine 2.5, |
| | | | | | | | | | | | | | | | | | | | HCTZ stopped, |
| | | | | | | | | | | | | | | | | | | | K+ 5.8 |
| Chol Rx | NO | 289.5 | 36.5 | 197 | 150 | | | 150 | | 6.0 | | 6.0 | | 6.0 | | | | | Feels fine. |
| | | | | | | | | | | | | | | | | | | | B.P. 108/76 |
| | | 293.0 | 33.0 | 279 | 75 | | 75 | 75 | | 6.0 | | 6.0 | | 6.0 | | | | | |
| Athero . . . | | 289.25 | 36.8 | 311 | 75 | | 75 | 75 | | 6.0 | | 6.0 | | 6.0 | | | | | |
| | | 284.75 | 41.3 | 315 | 75 | | 75 | 75 | | 6.0 | | 6.0 | | 6.0 | | | | | |
| Diabetes 2 | NO | 287.75 | 38.3 | 336 | 75 | | 75 | 75 | | 6.0 | | 6.0 | | 6.0 | | | | | |

| | | | | | | | | | | |
|---|---|---|---|---|---|---|---|---|---|---|
| Diab. Rx | 287.50 | 38.5 | 363 | 150 | 0 | 150 | 6.0 | 6.0 | 6.0/3.0 | New Orleans |
| | 281.75 | 44.3 | 376 | 150 | 0 | 150 | 6.0 | 6.0 | 6.0/3.0 | |
| | 272.0 | 54.0 | 405 | | | | | | | Vomiting |

| Depress | 280.5 | 45.5 | 419 | 150 | 150/75 | 6.0 | 6.0 | 6.0/3.0 | |
|---|---|---|---|---|---|---|---|---|---|
| Depress Rx | 278.5 | 47.5 | 433 | 150 | 150/75 | 6.0 | 6.0 | 6.0/3.0 | |
| | 279.5 | 46.5 | 454 | 150 | 150/75 | 7.5 | 7.5 | 7.5 | |
| Medications | 279.0 | 47.0 | 468 | 150 | 150/75 | 7.5 | 7.5 | 7.5 | |
| HCTZ | 280.0 | 46.0 | 482 | 150 | 150/75 | 6.0 | 6.0 | 6.0 | Oatmeal |

| | | | | | | | | | | |
|---|---|---|---|---|---|---|---|---|---|---|
| Metoprolol | 276.0 | 50.0 | 498 | 150 | | 150/75 | 6.0 | 6.0 | 6.0/3.0 | |
| Nisoldipine | 273.8 | 52.2 | 510 | | | | | | | |
| Lisinopril | 272.8 | 53.2 | 530 | 150 | 150 | 150/75 | 6.0 | 6.0 | 6.0/3.0 | PIO @ |
| Allopurinol | 273.5 | 52.5 | 558 | 150 | 150 | 150/75 | 6.0 | 6.0 | 6.0/3.0 | Beer 24 CA |
| | | | | | | | | | | US 4D |
| Celecoxib | 272.8 | 53.2 | 579 | 150 | 150 | 150/75 | 6.0 | 6.0 | 6.0/3.0 | |
| Colchicine | | | | 150 | 150 | 225/0 | 6.0 | 6.0 | 9.0/0 | |

| | | | | | Venlafaxine | | | | | Rivastigmine | | | | | Traza- | | | |
|---|---|---|---|---|---|---|---|---|---|---|---|---|---|---|---|---|---|---|
| Patient | | | Wt. | | a | | a | a | | a | | a | a | | done | | Diethyl- | |
| O1 | | Weight | Chg. | Days | brkfast | | lunch | supper | | brkfast | | lunch | supper | | am | pm | propion | Comments |
| Height | 64.25 | 209.5 | 0.0 | 0 | | | | | | | | | | | | | | |
| BMI | 36.1 | 193.3 | 16.3 | 73 | 75 | mg | | 75 | mg | 1.5 | mg | | 1.5 | mg | | | | |
| Sex | F | 188.8 | 20.8 | 94 | 75 | | | 75 | | 1.5 | | | 1.5 | | | | | |
| Birth Date | Jul. 21, 1958 | 176.5 | 33.0 | 121 | 75 | | 75 | 75 | | 1.5 | | 1.5 | 1.5 | | | | | |
| Age at onset | 44 | 179.5 | 30.0 | 135 | 75 | | 75 | 75 | | 1.5 | | 1.5 | 1.5 | | | | | |
| Initial info. | | | | 149 | 75 | | 75 | 75 | | 1.5 | | 1.5 | 1.5 | | | | | |
| B.P. | 120/82 | 182.0 | 27.5 | 167 | 75 | | 75 | 75 | | 1.5 | | 1.5 | 1.5 | | | | | |
| Pulse | 75 | | | 171 | 75 | | 75 | 75 | | 1.5 | | 1.5 | 1.5 | | | | | |
| HPN Rx | YES | 180.0 | 29.5 | 222 | 75 | | 75 | 75 | | 3.0 | | 3.0 | 3.0 | | | | | |
| | | 177.5 | 32.0 | 235 | 75 | | 75 | 75 | | 1.5 | | 1.5 | 1.5 | | | | | |
| T. Chol. | 194 | | | | | | | | | | | | | | | | | |
| HDL | 39 | | | | | | | | | | | | | | | | | |
| TRG. | 104 | | | | | | | | | | | | | | | | | |
| Chol Rx | NO | | | | | | | | | | | | | | | | | |
| Athero . . . | | | | | | | | | | | | | | | | | | |
| Diabetes 2 | NO | | | | | | | | | | | | | | | | | |
| Diab. Rx | | | | | | | | | | | | | | | | | | |
| Depress | | | | | | | | | | | | | | | | | | |
| Depress Rx | | | | | | | | | | | | | | | | | | |
| Medications | | | | | | | | | | | | | | | | | | |
| HCTZ | | | | | | | | | | | | | | | | | | |
| Lisinopril | | | | | | | | | | | | | | | | | | |
| Amlodipine | | | | | | | | | | | | | | | | | | |
| Vicodin | | | | | | | | | | | | | | | | | | |

| | | | | | Venlafaxine | | | | | Rivastigmine | | | | | Traza- | | | |
|---|---|---|---|---|---|---|---|---|---|---|---|---|---|---|---|---|---|---|
| Patient | | | | | a | | a | a | | a | | a | a | | done | | Diethyl- | |
| R1 | | Weight | Δ Wt. | Days | brkfast | | lunch | supper | | brkfast | | lunch | supper | | am | pm | propion | Comments |
| Height | 71.25 | 288.8 | 0.0 | 0 | | | | | | | | | | | | | | Protein “C” and |
| | | | | | | | | | | | | | | | | | | “S” deficiency, |
| | | | | | | | | | | | | | | | | | | Gout |
| BMI | 40.1 | 288.0 | 0.8 | 2 | 75 | mg | | 75 | mg | 1.5 | mg | | 1.5 | mg | | | | Peak wt. 334 in |
| | | | | | | | | | | | | | | | | | | Oct. 16, 1997, |
| | | | | | | | | | | | | | | | | | | low wt. 266 |
| | | | | | | | | | | | | | | | | | | in May 21, |
| | | | | | | | | | | | | | | | | | | 1999; lowest |
| | | | | | | | | | | | | | | | | | | since '85 |
| Sex | M | 284.0 | 4.8 | 21 | 75 | | | 75 | | 1.5 | | | 1.5 | | | | | |
| Birth | Jul. 17, | 276.8 | 12.0 | 34 | 75 | | | 75 | | 1.5 | | | 1.5 | | | | | Lethargy |
| Date | 1946 | | | | | | | | | | | | | | | | | |
| Age at | 56 | 273.5 | 15.3 | 48 | 75 | | | 75 | | 1.5 | | | 1.5 | | | | | Orthostatic |
| onset | | | | | | | | | | | | | | | | | | Hypotension |
| Initial | | 275.5 | 13.3 | 68 | 75 | | | 75 | | 1.5 | | | 1.5 | | | | | |
| info. | | | | | | | | | | | | | | | | | | |
| B.P. | 114/74 | 270.5 | 18.3 | 83 | 75 | | | 75 | | 3.0 | | | 3.0 | | | | | |
| Pulse | 60 | 269.25 | 19.6 | 99 | 75 | | | 75 | | 3.0 | | | 3.0 | | | | | PIO since 3/03 |
| HPN Rx | NO | 264.25 | 24.6 | 111 | 75 | | 75 | 75 | | 3.0 | | 3.0 | 3.0 | | | | | |
| | | 260.50 | 28.3 | 125 | 75 | | 75 | 75 | | 3.0 | | 3.0 | 3.0 | | | | | |
| T. Chol. | 164 | 270.0 | 18.8 | 139 | 75 | | 75 | 75 | | 3.0 | | 3.0 | 3.0 | | | | | France |
| HDL | 51 | 260.5 | 28.3 | 153 | 75 | | 75 | 75 | | 3.0 | | 3.0 | 3.0 | | | | | Meds later |
| | | | | | | | | | | | | | | | | | | in day |
| TRG. | 70 | 261.5 | 27.3 | 167 | 75 | | 75 | 75 | | 3.0 | | 3.0 | 3.0 | | | | | |
| Chol Rx | YES | 261.75 | 27.1 | 181 | 75 | | 75 | 75 | | 3.0 | | 3.0 | 3.0 | | | | | |
| | | 257.25 | 31.6 | 195 | | | | | | | | | | | | | | |
| Athero . . . | | 263.25 | 25.6 | 210 | | | | | | | | | | | | | | |
| | | 260.5 | 28.3 | 223 | 75 | | P75 | P75 | | 3.0 | | P3.0 | P3.0 | | | | | P = post meal |
| Diabetes | YES | 255.0 | 33.8 | 237 | 75 | | 75 | P150 | | 3.0 | | P3.0 | P4.5 | | | | | |
| 2 | | | | | | | | | | | | | | | | | | |
| Diab. Rx | YES | 256.2 | 32.6 | 251 | 75 | | P75 | P150 | | 3.0 | | P3.0 | P4.5 | | | | | Dinner 4-18 |
| | | 254.5 | 34.3 | 272 | 75 | | P75 | P150 | | 3.0 | | P3.0 | P4.5 | | | | | |
| Depress | | 257.0 | 31.8 | 286 | 75 | | P75 | P150 | | 3.0 | | P3.0 | P4.5 | | | | | |
| Depress Rx | | 256.5 | 32.3 | 300 | 75 | | P75 | P150 | | 3.0 | | P3.0 | P4.5 | | | | | |
| | | 257.0 | 31.8 | 314 | 75 | | P75 | P150 | | 3.0 | | P3.0 | P6.0 | | | | | |
| Medications | | 259.5 | 29.3 | 328 | 75 | | P75 | P150 | | 3.0 | | P3.0 | P6.0 | | | | Delay | |
| Warfarin | | 253.8 | 35.0 | 342 | 75 | | P150 | P150 | | 3.0 | | P3.0 | P6.0 | | | | 50/10 pm | |
| Metformin | | | | | | | | | | | | | | | | | | |
| Pioglitasone | | | | | | | | | | | | | | | | | | |
| Allopurinol | | | | | | | | | | | | | | | | | | |
| Colchicene | | | | | | | | | | | | | | | | | | |
| Pravastatin | | | | | | | | | | | | | | | | | | |
| Nisapan | | | | | | | | | | | | | | | | | | |
| Flu vaccine | | | | | | | | | | | | | | | | | | |

| | | | | | Venlafaxine | | | | | Rivastigmine | | | | | Traza- | | | | |
|---|---|---|---|---|---|---|---|---|---|---|---|---|---|---|---|---|---|---|---|
| Patient | | | Wt. | | a | | a | a | | a | | a | a | | done | | Diethyl- | | |
| R2 | | Weight | Chg. | Days | brkfast | | lunch | supper | | brkfast | | lunch | supper | | am | pm | propion | | Comments |
| Height | 68.50 | 209.3 | 0.0 | 0 | | | | | | | | | | | | | | | WBC 13.0, |
| | | | | | | | | | | | | | | | | | | | Fibrinogen |
| | | | | | | | | | | | | | | | | | | | 715, Donepezil |
| | | | | | | | | | | | | | | | | | | | for 1 month |
| BMI | 31.4 | 207.5 | 1.8 | 14 | 75 | mg | | | | | | | | | | | | | |
| Sex | M | 206.0 | 3.3 | 35 | 75 | | | | | | | | | | | | | | |
| Birth | Oct. 15, | 213.3 | −4.0 | 111 | 75 | | | | | | | | | | | | | | |
| Date | 1948 | | | | | | | | | | | | | | | | | | |
| Age at | 54 | 211.3 | −2.0 | 132 | 75 | | | 75 | mg | 1.5 | mg | | 1.5 | mg | | | | | |
| onset | | | | | | | | | | | | | | | | | | | |
| Initial | | 208.8 | 0.5 | 167 | 75 | | | 75 | | 1.5 | | | 3.0 | | | | | | |
| info. | | | | | | | | | | | | | | | | | | | |
| B.P. | 132/70 | 212.0 | −2.7 | 203 | 75 | | | 75 | | 1.5 | | | 1.5 | | | | | | |
| Pulse | 64 | 210.5 | −1.2 | 239 | 150 | | | 75 | | 1.5 | | | 1.5 | | | | | | |
| HPN Rx | YES | 209.0 | 0.3 | 251 | 75 | | | 75 | | 1.5 | | | 1.5 | | | | | | |
| | | 209.0 | 0.3 | 272 | 75 | | | 75 | | 3.0 | | | 3.0 | | | | | | |
| T. Chol. | 183 | 209.2 | 0.1 | 286 | 75 | | | 75 | | 3.0 | | | 3.0 | | | | | | |
| HDL | 43 | 210.5 | −1.2 | 301 | 75 | | | 75 | | 3.0 | | | 3.0 | | | | 25 | 25 | |
| TRG. | 156 | 213.0 | −3.7 | 314 | 75 | | | 75 | | 3.0 | | | 3.0 | | | | 25 | 25 | |
| Chol Rx | YES | 209.8 | −0.5 | 398 | 75 | | | 75 | | 4.5 | | 4.5 | 4.5 | | | | 25 | 25 | |
| | | 211.2 | −1.9 | 417 | | | | | | 0.0 | | | 0.0 | | | | | | Trying to |
| | | | | | | | | | | | | | | | | | | | lose weight |
| Athero . . . | | 210.5 | −1.2 | 431 | | | | 0 | | 0.0 | | | 0.0 | | | | | | on his own |
| | | 212.25 | −2.9 | 448 | | | | 0 | | 0.0 | | | 0.0 | | | | | | |
| Diabetes | NO | 209.5 | −0.2 | 455 | | | | | | | | | | | | | | | |
| 2 | | | | | | | | | | | | | | | | | | | |
| Diab. Rx | | | | | | | | | | | | | | | | | | | |
| Depress | | | | | | | | | | | | | | | | | | | |
| Depress Rx | NO | | | | | | | | | | | | | | | | | | |
| Medications | | | | | | | | | | | | | | | | | | | |
| HCTZ | | | | | | | | | | | | | | | | | | | |
| Lisinopril | | | | | | | | | | | | | | | | | | | |
| Nisoldipine | | | | | | | | | | | | | | | | | | | |
| Metoprolol | | | | | | | | | | | | | | | | | | | |
| Pravastatin | | | | | | | | | | | | | | | | | | | |
| Sildenafil | | | | | | | | | | | | | | | | | | | |

| | | | | | Venlafaxine | | | | | Rivastigmine | | | | | Traza- | | | |
|---|---|---|---|---|---|---|---|---|---|---|---|---|---|---|---|---|---|---|
| Patient | | | Wt. | | a | a | | a | | a | a | | a | | done | | Diethyl- | |
| T1 | | Weight | Chg. | Days | brkfast | lunch | | supper | | brkfast | lunch | | supper | | am | pm | propion | Comments |
| Height | — | 140.0 | 0.0 | 0 | | | | | | | | | | | | | | |
| BMI | | 139.0 | 1.0 | 5 | | 37.5 | mg | 37.5 | mg | | 1.5 | mg | 1.5 | mg | | | | Nausea, |
| | | | | | | | | | | | | | | | | | | treatment |
| | | | | | | | | | | | | | | | | | | by phone |
| Sex | F | 138.0 | 2.0 | 19 | | 37.5 | | 37.5 | | | 1.5 | | 1.5 | | | | | Nausea |
| Birth Date | Oct. 25, | 140.2 | −0.2 | 30 | | 37.5 | | 75 | | | 2.25 | | 2.25 | | | | | |
| | 1970 | | | | | | | | | | | | | | | | | |
| Age at | 32 | 136.5 | 3.5 | 41 | | 37.5 | | 75 | | | 1.5 | | 1.5 | | | | | |
| onset | | | | | | | | | | | | | | | | | | |
| Initial | | | | | | | | | | | | | | | | | | |
| info. | | | | | | | | | | | | | | | | | | |
| B.P. | — | | | | | | | | | | | | | | | | | |
| Pulse | — | | | | | | | | | | | | | | | | | |
| HPN Rx | NO | | | | | | | | | | | | | | | | | |
| T. Chol. | — | | | | | | | | | | | | | | | | | |
| HDL | — | | | | | | | | | | | | | | | | | |
| TRG. | — | | | | | | | | | | | | | | | | | |
| Chol Rx | | | | | | | | | | | | | | | | | | |
| Athero . . . | | | | | | | | | | | | | | | | | | |
| Diabetes 2 | | | | | | | | | | | | | | | | | | |
| Diab. Rx | YES | | | | | | | | | | | | | | | | | |
| Depress | | | | | | | | | | | | | | | | | | |
| Depress Rx | | | | | | | | | | | | | | | | | | |
| Medications | | | | | | | | | | | | | | | | | | |

| | | | | | Venlafaxine | | | | | Rivastigmine | | | | | Traza- | | | |
|---|---|---|---|---|---|---|---|---|---|---|---|---|---|---|---|---|---|---|
| Patient | | | Wt. | | a | | a | a | | a | | a | a | | done | | Diethyl- | |
| T2 | | Weight | Chg. | Days | brkfast | | lunch | supper | | brkfast | | lunch | supper | | am | pm | propio [symbol] | Comments |
| Height | 66.25 | 210.0 | 0.0 | 0 | | | | | | | | | | | | | | Was on |
| | | | | | | | | | | | | | | | | | | Trazodone and |
| | | | | | | | | | | | | | | | | | | Paroxetine |
| BMI | 30.8 | 190.5 | 19.5 | 63 | 75 | mg | | 75 | mg | 1.5 | mg | | 1.5 | mg | | | | |
| Sex | F | 187.8 | 22.3 | 92 | 75 | | | 75 | | 1.5 | | | 1.5 | | | | | |
| Birth Date | Jun. 10, 1947 | 180.0 | 30.0 | 240 | 75 | | | 75 | | 1.5 | | | 1.5 | | | | | |
| Age at onset | 55 | | | | | | | | | | | | | | | | | Lost interest |
| Initial info. | | | | | | | | | | | | | | | | | | |
| B.P. | 122/72 | | | | | | | | | | | | | | | | | |
| Pulse | 82 | | | | | | | | | | | | | | | | | |
| HPN Rx | YES | | | | | | | | | | | | | | | | | |
| T. Chol. | 186 | | | | | | | | | | | | | | | | | |
| HDL | 45 | | | | | | | | | | | | | | | | | |
| TRG. | 208 | | | | | | | | | | | | | | | | | |
| Chol Rx | YES | | | | | | | | | | | | | | | | | |
| Athero . . . | | | | | | | | | | | | | | | | | | |
| Diabetes 2 | NO | | | | | | | | | | | | | | | | | |
| Diab. Rx | | | | | | | | | | | | | | | | | | |
| Depress | YES | | | | | | | | | | | | | | | | | |
| Depress Rx | YES | | | | | | | | | | | | | | | | | |
| Medications | | | | | | | | | | | | | | | | | | |

| | | | | | Venlafaxine | | | | | | Rivastigmine | | | | | Traza- | | | |
|---|---|---|---|---|---|---|---|---|---|---|---|---|---|---|---|---|---|---|---|
| Patient | | | | | a | | a | | a | | a | a | | a | | done | | Diethyl- | |
| 2/E-R | | Weight | Δ Wt. | Days | brkfast | | lunch | | supper | | brkfast | lunch | | supper | | am | pm | propion | Comments |
| Height | 65 | 310.75 | 0.0 | 0 | | | | | | | | | | | | | | | |
| BMI | 51.8 | 309.00 | 1.75 | 13 | 75 | mg | | | 75 | mg | | 1.5 | mg | 1.5 | mg | | | | |
| Sex | M | 307.25 | 3.50 | 27 | | | 75 | mg | 75 | | | 3.0 | | 3.0 | | | | | |
| Birth | | 298.75 | 12.00 | 41 | | | 150 | | 150 | | | 3.0 | | 3.0 | | | | | |
| Date | | | | | | | | | | | | | | | | | | | |
| Age at | 48 | 293.5 | 17.25 | 55 | | | 150 | | 150 | | | 3.0 | | 3.0 | | | | | |
| onset | | | | | | | | | | | | | | | | | | | |
| Initial | | 291.0 | 19.75 | 69 | | | 150 | | 150 | | | 3.0 | | 3.0 | | | | | |
| info. | | | | | | | | | | | | | | | | | | | |
| B.P. | 140/88 | 290.0 | 20.75 | 83 | | | 150 | | 150 | | | 3.0 | | 3.0 | | | | | |
| Pulse | 110 | 288.0 | 22.75 | 97 | | | 150/150 | | 150 | | | 3.0/3.0 | | 3.0 | | | | | |
| HPN Rx | | 280.8 | 30.00 | 111 | | | 150/150 | | 150 | | | 3.0/3.0 | | 3.0 | | | | | |
| | | 278.8 | 32.00 | 125 | | | 150/150 | | 150 | | | 3.0/3.0 | | 3.0 | | | | | |
| T. Chol. | | 280.5 | 30.25 | 139 | | | 150/150 | | 150 | | | 3.0/3.0 | | 3.0 | | | | | |
| HDL | | 275.8 | 35.00 | 153 | | | 150/150 | | 150 | | | 3.0/3.0 | | 3.0 | | | | | |
| TRG. | | 272..5 | 38.25 | 167 | 75 | | 75 | | 75 | | | 3.0 | | 3.0 | | | | | |
| Chol Rx | | 271.5 | 39.25 | 182 | 75 | | 75 | | 75 | | | 3.0 | | 3.0 | | | | | |
| | | 271.5 | 39.25 | 195 | 75 | | 75 | | 75 | | | 3.0 | | 3.0 | | | | | |
| Athero . . . | | 271.2 | 39.55 | 209 | 75 | | 75 | | 75 | | 3.0 | 6.0 | | 3.0 | | | | | |
| | | 268.2 | 42.55 | 216 | 75 | | 75 | | 75 | | 3.0 | 6.0 | | 3.0 | | | | | |
| Diabetes | | | | | | | | | | | | | | | | | | | |
| 2 | | | | | | | | | | | | | | | | | | | |
| Diab. Rx | | | | | | | | | | | | | | | | | | | |
| Depress | | | | | | | | | | | | | | | | | | | |
| Depress Rx | | | | | | | | | | | | | | | | | | | |
| Medications | | | | | | | | | | | | | | | | | | | |

| | | | | | Venlafaxine | | | | Rivastigmine | | | Traza- | | | |
|---|---|---|---|---|---|---|---|---|---|---|---|---|---|---|---|
| Patient | | | | | a | a | a | | a | a | a | done | | Diethyl- | |
| 6/B-G | | Weight | Δ Wt. | Days | brkfast | lunch | supper | | brkfast | lunch | supper | am | pm | propion | Comments |
| Height | | 259.2 | 0.0 | 0 | | | | | | | | | | | |
| BMI | | 242.5 | 16.7 | 13 | | | 150 | mg | | | 1.5 | | | | |
| Sex | | 241.5 | 17.7 | 20 | | | 150 | | | | 1.5 | | | | |
| Birth Date | | 237.5 | 21.7 | 28 | | | 150 | | | | 1.5 | | | | |
| Age at onset | 57 | 237.5 | 21.7 | 42 | | | 150 | | | | 1.5 | | | | |
| Initial info. | | 235.0 | 24.2 | 56 | | | 150 | | | | 1.5 | | | | |
| B.P. | | | | | | | 150 | | | | 3.0 | | | | |
| Pulse | | | | | | | 225 | | | | 4.5 | | | | |
| HPN Rx | | | | | | | | | | | | | | | |
| T. Chol. | | | | | | | | | | | | | | | |
| HDL | | | | | | | | | | | | | | | |
| TRG. | | | | | | | | | | | | | | | |
| Chol Rx | | | | | | | | | | | | | | | |
| Athero . . . | | | | | | | | | | | | | | | |
| Diabetes 2 | | | | | | | | | | | | | | | |
| Diab. Rx | | | | | | | | | | | | | | | |
| Depress | | | | | | | | | | | | | | | |
| Depress Rx | | | | | | | | | | | | | | | |
| Medications | | | | | | | | | | | | | | | |

| | | | | | Venlafaxine | | | | Rivastigmine | | | | Traza- | | | |
|---|---|---|---|---|---|---|---|---|---|---|---|---|---|---|---|---|
| Patient | | | | | a | a | a | | a | a | a | | done | | Diethyl- | |
| 8/P-B | | Weight | Δ Wt. | Days | brkfast | lunch | supper | | brkfast | lunch | supper | | am | pm | propion | Comments |
| Height | | 251.5 | 0.0 | 0 | | | | | | | | | | | | |
| BMI | | 246.5 | 5.0 | 14 | | | 75 | mg | | | 1.5 | mg | | | | |
| Sex | M | 241.8 | 9.7 | 28 | | | | | | | | | | | | |
| Birth Date | | 241.8 | 9.7 | 42 | | | | | | | | | | | | |
| Age at onset | 46 | 241.8 | 9.7 | 56 | | | 75 | | | | 1.5 | | | | | |
| Initial info. | | 243.8 | 7.7 | 70 | | | 75 | | | | 1.5 | | | | | |
| B.P. | | 240.8 | 10.7 | 84 | | 75 | 150 | | | | 1.5 | | | | | |
| Pulse | | | | | | | | | | | | | | | | |
| HPN Rx | | | | | | | | | | | | | | | | |
| T. Chol. | | | | | | | | | | | | | | | | |
| HDL | | | | | | | | | | | | | | | | |
| TRG. | | | | | | | | | | | | | | | | |
| Chol Rx | | | | | | | | | | | | | | | | |
| Athero . . . | | | | | | | | | | | | | | | | |
| Diabetes 2 | | | | | | | | | | | | | | | | |
| Diab. Rx | | | | | | | | | | | | | | | | |
| Depress | | | | | | | | | | | | | | | | |
| Depress Rx | | | | | | | | | | | | | | | | |
| Medications | | | | | | | | | | | | | | | | |

| | | | | | Venlafaxine | | | | | Rivastigmine | | | | | Traza- | | | |
|---|---|---|---|---|---|---|---|---|---|---|---|---|---|---|---|---|---|---|
| Patient | | | | | a | | a | a | | a | | a | a | | done | | Diethyl- | |
| 11/A-S | | Weight | Δ Wt. | Days | brkfast | | lunch | supper | | brkfast | | lunch | supper | | am | pm | propion | Comments |
| Height | 67.5 | 165.0 | 0.0 | 0 | | | | | | | | | | | | | | |
| BMI | 24.5 | 159.0 | 6.0 | 14 | 9 | mg | | 9 | mg | 0.4 | mg | | 0.4 | mg | | | | By phone only |
| Sex | | 155.0 | 10.0 | 28 | 18 | | | 18 | | 0.8 | | | 0.8 | | | | | |
| Birth Date | F | 155.0 | 10.0 | 41 | 9 | | | 9 | | 0.8 | | | 0.8 | | | | | |
| Age at onset | 32 | 154.0 | 11.0 | 56 | 11 | | | 11 | | 1.5 | | | 1.5 | | | | | |
| Initial info. | | 156.0 | 9.0 | 87 | 19 | | | 19 | | 1.5 | | | 1.5 | | | | | |
| B.P. | | 154.0 | 11.0 | 97 | 75 | | | 75 | | 1.5 | | | 1.5 | | | | | |
| Pulse | | 151.0 | 14.0 | 116 | 75 | | | 75 | | 1.5 | | | 1.5 | | | | | |
| HPN Rx | | 150.0 | 15.0 | 126 | 75 | | | 75 | | 1.5 | | | 1.5 | | | | | |
| | | | | | 75 | | | 75 | | 3.0 | | | 3.0 | | | | | Tonsil surgery |
| T. Chol. | | | | | | | | | | | | | | | | | | |
| HDL | | | | | | | | | | | | | | | | | | |
| TRG. | | | | | | | | | | | | | | | | | | |
| Chol Rx | | | | | | | | | | | | | | | | | | |
| Athero . . . | | | | | | | | | | | | | | | | | | |
| Diabetes 2 | | | | | | | | | | | | | | | | | | |
| Diab. Rx | | | | | | | | | | | | | | | | | | |
| Depress | | | | | | | | | | | | | | | | | | |
| Depress Rx | | | | | | | | | | | | | | | | | | |
| Medications | | | | | | | | | | | | | | | | | | |
| HCTZ | | | | | | | | | | | | | | | | | | |
| Lisinopril | | | | | | | | | | | | | | | | | | |
| Nisoldipine | | | | | | | | | | | | | | | | | | |
| Metoprolol | | | | | | | | | | | | | | | | | | |
| Pravastatin | | | | | | | | | | | | | | | | | | |
| Sildenafil | | | | | | | | | | | | | | | | | | |
| Bacitracin | | | | | | | | | | | | | | | | | | |
| aderall | | | | | | | | | | | | | | | | | | |

| | | | | | Venlafaxine | | | | Rivastigmine | | | | Traza- | | | |
|---|---|---|---|---|---|---|---|---|---|---|---|---|---|---|---|---|
| Patient | | | | | a | a | a | | a | a | a | | done | | Diethyl- | |
| 13/L-V | | Weight | Δ Wt. | Days | brkfast | lunch | supper | | brkfast | lunch | supper | | am | pm | propion | Comments |
| Height | 71.2 | 274.8 | 0.0 | 0 | | | | | | | | | | | | |
| BMI | 38.1 | 268.2 | 6.6 | 14 | | | 75 | XR | | | 1.5 | mg | | | | |
| Sex | M | 264.0 | 10.8 | 21 | | | 75 | IR | | | 1.5 | | | | | |
| Birth Date | | 262.0 | 12.8 | 32 | | | 75 | | | | 1.5 | | | | | |
| Age at onset | 63 | 265.5 | 9.3 | 39 | | | 75 | | | | 1.5 | | | | | |
| Initial info. | | 263.2 | 11.6 | 53 | | | 75 | | | | 1.5 | | | | | |
| B.P. | 120/78 | 260.8 | 14.0 | 68 | | | 75 | | | | 1.5 | | | | | |
| Pulse | 64 | 263.5 | 11.3 | 81 | | | 75 | | | | 1.5 | | | | | |
| HPN Rx | YES | 254.0 | 20.8 | 95 | | | 150 | | | | 3.0 | | | | | |
| | | 258.0 | 16.8 | 109 | | | 150 | | | | 3.0 | | | | | G.I. Bleed 7-13 |
| T. Chol. | | 252.0 | 22.8 | 123 | | | 150 | | | | 3.0 | | | | | PIO Stopped |
| HDL | | | | | | | | | | | | | | | | |
| TRG. | | | | | | | | | | | | | | | | |
| Chol Rx | YES | | | | | | | | | | | | | | | |
| Athero . . . | | | | | | | | | | | | | | | | |
| Diabetes 2 | | | | | | | | | | | | | | | | |
| Diab. Rx | | | | | | | | | | | | | | | | |
| Depress | | | | | | | | | | | | | | | | |
| Depress Rx | | | | | | | | | | | | | | | | |
| Medications | | | | | | | | | | | | | | | | |
| Lisinopril | | | | | | | | | | | | | | | | |
| Atenolol | | | | | | | | | | | | | | | | |
| Pioglitazone | | | | | | | | | | | | | | | | |
| Pravastatin | | | | | | | | | | | | | | | | |
| Verapamil | | | | | | | | | | | | | | | | |

1. A method of treating obesity, said method comprising administering to a subject in need thereof, an effective amount of a combination of one or more cholinesterase inhibitors and one or more antidepressants, whereby said obesity is treated.

2. The method in accordance with claim 1, wherein said one or more cholinesterase inhibitors is selected from the group consisting of a reversible cholinesterase inhibitor, a pseudo-irreversible cholinesterase inhibitor, an irreversible cholinesterase inhibitor, and mixtures thereof.

3. The method in accordance with claim 2, wherein said one or more reversible cholinesterase inhibitors is selected from the group consisting of tacrine, donepezil, edrophonium, galantamine, and mixtures thereof.

4. The method in accordance with claim 2, wherein said one or more pseudo-irreversible cholinesterase inhibitors is selected from the group consisting of physostigmine, eptastigmine, pyridostigmine, neostigmine, ganstigmine, rivastigmine, demecarium, ambenonium and mixtures thereof.

5. The method in accordance with claim 2, wherein said one or more irreversible cholinesterase inhibitors comprises an organophosphate.

6. The method in accordance with claim 2, wherein said one or more irreversible cholinesterase inhibitors is selected from the group consisting of sarin, metrifonate, soman, tabun, diisopropyl fluorophosphate, and mixtures thereof.

7. The method in accordance with claim 1, wherein said one or more antidepressants is selected from the group consisting of tricyclic antidepressants and analogs thereof, serotonin reuptake inhibitors, serotonin-norepinephrine reuptake inhibitors, norepinephrine reuptake inhibitors, dopamine reuptake inhibitors, norepinephrine-dopamine reuptake inhibitors, serotonin-norepinephrine-dopamine reuptake inhibitors, serotonin reuptake accelerators, serotonin agonists and prodrugs thereof, monoamine oxidase inhibitors and mixtures thereof.

8. The method in accordance with claim 7, wherein said tricyclic antidepressant and analogs thereof are selected from the group consisting of amineptine, amitriptyline, clomipramine, desipramine, doxepin, dothiepin, imipramine, nortriptyline, protriptyline, trimipramine, amoxapine, maprotiline, cyclobenzaprine and mixtures thereof.

9. The method in accordance with claim 7, wherein said serotonin reuptake inhibitors are selective serotonin reuptake inhibitors selected from the group consisting of citalopram, escitalopram, fluoxetine, fluvoxamine, paroxetine, sertraline, and mixtures thereof.

10. The method in accordance with claim 7, wherein said serotonin-norepinephrine reuptake inhibitors are selected from the group consisting of milnacipran, mirtazapine, venlafaxine, duloxetine, S33005, DVS-233 (desvenlafaxine), DVS-233 SR, sibutramine and mixtures thereof.

11. The method in accordance with claim 7, wherein said norepinephrine reuptake inhibitors are selective norepinephrine reuptake inhibitors selected from the group consisting of reboxetine, atomoxetine and mixtures thereof.

12. The method in accordance with claim 7, wherein said norepinephrine-dopamine reuptake inhibitors are selected from the group consisting of amineptine, bupropion, GW353162 and mixtures thereof.

13. The method in accordance with claim 7, wherein said monoamine oxidase inhibitors are selected from the group consisting of befloxatone, brofaromine, deprenyl, isocarboxazid, moclobemide, pargyline, phenelzine, selegiline, tranylcypromine and mixtures thereof.

14. The method in accordance with claim 1, comprising administering a combination of effective amounts of a cholinesterase inhibitor and a selective serotonin reuptake inhibitor.

15. The method in accordance with claim 14, wherein said combination comprises effective amounts of galantamine and citalopram.

16. The method in accordance with claim 15, wherein said galantamine is administered in an amount of 4 mg/dose and said citalopram is administered in an amount of 20 mg/dose.

17. The method in accordance with claim 15, wherein said galantamine and said citalopram are administered once per day.

18. The method in accordance with claim 14, wherein said combination comprises effective amounts of donepezil and sertraline.

19. The method in accordance with claim 1, comprising administering a combination of effective amounts of a cholinesterase inhibitor and a serotonin-norepinephrine reuptake inhibitor.

20. The method in accordance with claim 19, wherein said combination comprises effective amounts of rivastigmine and venlafaxine.

21. The method in accordance with claim 20, wherein said rivastigmine is administered in an amount of 0.4-6.0 mg/dose and said venlafaxine is administered in an amount of 37.5-225 mg/dose.

22. The method in accordance with claim 20, wherein said rivastigmine and said venlafaxine are administered twice per day.

23. The method in accordance with claim 1, wherein said cholinesterase inhibitor is selected from the group consisting of rivastigmine, galantamine and donepezil and said antidepressant is selected from the group consisting of venlafaxine, citalopram, escitalopram, fluvoxamine, paroxetine, duloxetine, sertraline, bupropion, GW353162, S33005, DVS-233 (desvenlafaxine), DVS-233 SR, and mixtures thereof.

24. The method in accordance with claim 1, wherein said cholinesterase inhibitor and said antidepressant are administered at the same time.

25. The method in accordance with claim 1, wherein said combination is administered in a controlled-release formulation.

26. The method in accordance with claim 1, wherein said cholinesterase inhibitor and said antidepressant are administered at different times.

27. The method in accordance with claim 1, wherein said subject loses at least about 15 pounds after about 70 days of treatment.

28. The method in accordance with claim 1, wherein said subject loses at least about 20 pounds after about 100 days of treatment.

29. The method in accordance with claim 1, further comprising administering an effective amount of an anorexiant.

30. The method in accordance with claim 29, wherein said anorexiant is selected from the group consisting of amphetamine, methamphetamine, dextroamphetamine, phentermine, benzphetamine, phendimetrazine, phenmetrazine, diethylpropion, mazindol, fenfluramine, phenylpropanolamine and mixtures thereof.

31. A method of achieving desirable weight loss, said method comprising administering to a subject in need thereof, an effective amount of a combination of one or more cholinesterase inhibitors and one or more antidepressants, whereby said subject loses a desirable amount of weight.

32. A method of preventing undesirable weight gain, said method comprising administering to a subject in need thereof, an effective amount of a combination of one or more cholinesterase inhibitors and one or more antidepressants, whereby said subject is prevented from gaining an undesirable amount of weight.

33. The method in accordance with claim 31 or claim 32, wherein said subject is overweight.

34. The method in accordance with claim 31 or claim 32, wherein said subject is obese.

35. A method of facilitating weight loss in an individual not suffering from depression, said method comprising administering to said individual an amount of a combination of one or more cholinesterase inhibitors and one or more antidepressants sufficient to effectuate weight loss.

36. A method of assisting weight loss in an individual in need thereof, the method comprising administering to said individual over a sustained period of time an amount of a combination of one or more cholinesterase inhibitors and one or more antidepressants sufficient to assist in weight loss.

37. A method of maintaining a stable weight in an individual, said method comprising administering to said individual an effective amount of a combination of one or more cholinesterase inhibitors and one or more antidepressants, whereby said individual maintains a stable weight.

38. The method in accordance with claim 37, wherein said individual is obese.

39. A method of reducing body weight in an individual in need thereof, the method comprising administering to said individual over a sustained period of time an amount of a combination of one or more cholinesterase inhibitors and one or more antidepressants sufficient to cause reduction in body weight in said individual.

40. A pharmaceutical composition comprising a mixture of effective amounts of one or more cholinesterase inhibitors and one or more antidepressants.

41. The pharmaceutical composition of claim 40, wherein said one or more cholinesterase inhibitors is selected from the group consisting of a reversible cholinesterase inhibitor, a pseudo-irreversible cholinesterase inhibitor, an irreversible cholinesterase inhibitor, and mixtures thereof.

42. The pharmaceutical composition of claim 40, wherein said one or more reversible cholinesterase inhibitors is selected from the group consisting of tacrine, donepezil, edrophonium, galantamine, and mixtures thereof.

43. The pharmaceutical composition of claim 40, wherein said one or more pseudo-irreversible cholinesterase inhibitors is selected from the group consisting of physostigmine, eptastigmine, pyridostigmine, neostigmine, ganstigmine, rivastigmine, demecarium, ambenonium and mixtures thereof.

44. The pharmaceutical composition of claim 40, wherein said one or more irreversible cholinesterase inhibitors comprises an organophosphate.

45. The pharmaceutical composition of claim 40, wherein said one or more irreversible cholinesterase inhibitors is selected from the group consisting of sarin, metrifonate, soman, tabun, diisopropyl fluorophosphate, and mixtures thereof.

46. The pharmaceutical composition of claim 40, wherein said one or more antidepressants is selected from the group consisting of tricyclic antidepressants and analogs thereof, serotonin reuptake inhibitors, serotonin-norepinephrine reuptake inhibitors, norepinephrine reuptake inhibitors, dopamine reuptake inhibitors, norepinephrine-dopamine reuptake inhibitors, serotonin-norepinephrine-dopamine reuptake inhibitors, serotonin reuptake accelerators, serotonin agonists and prodrugs thereof, and mixtures thereof.

47. The pharmaceutical composition of claim 40, wherein said one or more cholinesterase inhibitors is selected from the group consisting of rivastigmine, galantamine, and donepezil, and said one or more antidepressants is selected from the group consisting of venlafaxine, citalopram, escitalopram, fluvoxamine, paroxetine, duloxetine, sertraline, bupropion, S33005, DVS-233 (desvenlafaxine), DVS-233 SR, and mixtures thereof.

48. The pharmaceutical composition of claim 40, wherein said one or more cholinesterase inhibitors comprises rivastigmine and said one or more antidepressants comprises venlafaxine.

49. The pharmaceutical composition of claim 40, wherein said one or more cholinesterase inhibitors comprises galantamine and said one or more antidepressants comprises citalopram.

50. The pharmaceutical composition of claim 40, wherein said one or more cholinesterase inhibitors comprises donepezil, and said one or more antidepressants comprises sertraline.

51. The pharmaceutical composition of claim 40, wherein said one or more cholinesterase inhibitors comprises galantamine and said one or more antidepressants comprises duloxetine.

52. The pharmaceutical composition of claim 40, wherein said one or more cholinesterase inhibitors comprises galantamine and said one or more antidepressants comprises paroxetine.

53. The pharmaceutical composition of claim 40, wherein said composition is a controlled-release composition.

54. A kit comprising a mixture of effective amounts of one or more cholinesterase inhibitors and one or more antidepressants.

55. The kit of claim 54, wherein said one or more cholinesterase inhibitors is selected from the group consisting of a reversible cholinesterase inhibitor, a pseudo-irreversible cholinesterase inhibitor, an irreversible cholinesterase inhibitor, and mixtures thereof.

56. The kit of claim 54, wherein said one or more antidepressants is selected from the group consisting of tricyclic antidepressants and analogs thereof, serotonin reuptake inhibitors, serotonin-norepinephrine reuptake inhibitors, norepinephrine reuptake inhibitors, dopamine reuptake inhibitors, norepinephrine-dopamine reuptake inhibitors, serotonin-norepinephrine-dopamine reuptake inhibitors, serotonin reuptake accelerators, serotonin agonists and prodrugs thereof, and mixtures thereof.

57. The kit of claim 56, wherein said one or more cholinesterase inhibitors is selected from the group consisting of rivastigmine, galantamine, and donepezil, and said one or more antidepressants is selected from the group consisting of venlafaxine, citalopram, escitalopram, fluvoxamine, paroxetine, duloxetine, sertraline, bupropion, GW353162, S33005, DVS-233 (desvenlafaxine), DVS-233 SR, and mixtures thereof.
